(19) United States
(12) Patent Application Publication
(10) Pub. No.: US 2004/0137551 A1
(43) Pub. Date: Jul. 15, 2004

(54) Cervical acid phosphatase - papanicolaou (CAP-PAP) test kit, method and accesories, processes for producing and using the same

(76) Inventors: Nenad S. Markovic, Rockville, MD (US); Olivera Markovic, Rockville, MD (US)

Correspondence Address: Dr. NENAD MARKOVIC, 14905 FOREST LANDING CIRCLES, ROCKVILLE, MD, 20850, US

(21) Appl. No.: 10/339,760

(22) Filed: Jan. 13, 2003

(51) Int. Cl. C12Q 1/44; C12Q 1/42

(52) U.S. Cl. 435/21

(57) ABSTRACT

Cervical Acid Phosphatase-Papanicolaou Test Kit (CPK) is an assembly of reagents, controls and instructions for visualization of cervical acid phosphatase on smears or monolayers of cervical specimens, and for performing the CAP-PAP Test (CPT).

CPT is a single-slide, double-staining method for demonstration of cervical acid phosphatase activity inside abnormal cervical cells on Papanicolaou stained smears, and a set of criteria for using this test for cervical cancer screening. In previous clinical trials this method was found to enable Pap test screeners to improve test sensitivity (detection of abnormal cells) for more than 10% (from 0.8 to 0.9), and to reduce false negative readings (missing abnormal cells) for more than 50% (from 0.1 to 0.02). Due to better accuracy and the low cost, when approved, CPT may begin to replace current technologies for cervical cancer screening.

CPK is designed to meet requirements for testing large series of specimens on regular basis—the usual practice in cytopathology laboratories performing the Pap test. CPK brings consistency for staining and interpretation, makes internal and external controls easier, and improves the test accuracy for lower cost, while increases laboratory productivity for less liability.

### 2.0 CROSS-REFERENCE TO OTHER APPLICATIONS

(For the complete list of references see the attached Literature Cited pages at the end of Specification).

2.1 Related Applications

This application claims benefit of the following prior applications by the same inventors.

2.1.1 Patent Documents by the Inventors

1. U.S. Pat. No. 6,143,512, “CAP-PAP TEST” UP issued on Nov. 7, 2000.

2. U.S. Ser. No. 09/329,445 “CAP-PAP TEST” UP filed Jul. 9, 1999.

3. U.S. Ser. No. 60/096744, filed Aug. 17, 1998.

4. U.S. Pat. No. 426850, ID “CAP-PAP Test for Cervical Cancer Screening” PP filed Oct. 24, 1997.

5. U.S. Pat. No. 487457, “CAP-PAP-Modified Test” ID filed Jan. 22, 2001.

6. U.S. Pat. No. 472,459, “ImmunoCAP”, ID filed Apr. 5, 2000.

7. U.S. Ser. No. 60/271,480, “Thin Layer Cervical Acid Phosphatase Papanicolaou Test” PP filed Feb. 27, 2001.

8. U.S. Pat. No. 504234, ID “CPT-SAS” ID filed Jan. 2, 2002.

9. U.S. Ser. No. 60/348,574 “Cervical Acid Phosphatase Papanicolaou Test (Research and Diagnostic) Kit, PP filed Jan. 16, 2002.

10. Markovic N, Markovic O. “Fructose ester-β-cyclodextrin” U.S. Pat. No. 5,620,961 of Apr. 15, 1997.

2.1.2 Other U.S. Patents

Only a single patent in the USPTO database is related to cervical acid phosphatase. This is our patent “CAP-PAP Test” that was issued on Nov. 7, 2000. However, between January 1996 (when we filed application for this patent) and Dec. 30, 2002 (when we completed patent literature search for the new application), there were issued 256 US patent related to the Pap test. Fewer patents were found under related queries. A total of 365 US patents related to Pap test, Pap smear, or vaginal/cervical examination, were found in the USPTO databases. A list of 43 patents most relevant to our disclosure(s) is provided on form pages “Information Disclosure, Statement by Applicant.”

2.1.3 International Patents

Majority of cited US patents bear International patent classification numbers. None of them was related to the cervical acid phosphatase.

2.1.4 FDA Database

According to the list of approved in vitro diagnostic medical devices, no one is based on cervical acid phosphatase as a biomarker for detection of abnormal cells signaling precancerous condition. Most of the devices related to Pap test and all of those included in this application, are cited by their manufacturers (See Web Page bibliography in the “Information Disclosure”).

2.2 Non-Patent Literature Cited

2.2.1 Grants and Grant Applications (See under U.S. Government Sponsored research)

2.2.2 Internet Web Sites

List of web links where appropriate literature is available.

2.2.3 Literature Cited

A list of the related literature references is provided on form pages “Information Disclosure, Statement by Applicant,” and in the special attachment at the end of this Specification.

### 3.0 STATEMENT REGARDING FEDERAL SPONSORED RESEARCH

This research was sponsored in part by Federal Government grants:

1. SBIR-NIH-NCI Grant No. 1 R43 CA 86767-01.

2. SBIR-NIH-NCI Grant No. 2 R44 CA 86767-02.

3. SBIR-NIH-NCI Grant No. 1 R43 CA 94628-01.

4. SBIR-NIH-NCI Application No. 1 R43 CA 101792-01.

### 4.0 BACKGROUND OF THE INVENTION

4.1 Scope of the Invention

According to the American Cancer Society, “Cervical cancer mortality has decreased over the last five decades by over 70 percent in large part attributable to the introduction of the Papanicolaou (Pap) test. Cervical cancer, once the number one cancer killer of women, now ranks 13^{th} in cancer deaths for women in the United States. As cervical cytology screening has become more prevalent, pre-invasive lesions of the cervix are detected more frequently than invasive cancers. In 2002, an estimated 13,000 cases of invasive cervical cancer will be diagnosed, and an estimated 4,100 women will die from this disease. Women with pre-invasive lesions have a five-year survival rate of nearly 100 percent. When cervical cancers are detected at an early stage, the five-year survival rate is approximately 92 percent.”^{78,86} Therefore, a pursuit for a more accurate test (less false-negatives) is a quest for saving lives.

About 50 million Pap tests are performed each year in the United States. Only 7 percent, or 3.5 million, are having cellular abnormalities requiring further investigation. Among these 7 percent two million are diagnosed as ASC-US and ASC-H, 1.25 million LSIL, and 300,000 HSIL in addition to 13,000 cervical cancer.⁸⁶ Obviously, majority of conditions presenting with cytological abnormalities of lower grades are reversible and do not progress to cervical cancer. False negatives are hidden among millions of women who were declared as negative/normal, or who did not have Pap test that year. The National Breast and Cervical Cancer Early Detection Program (NBCCEDP) reported that a women found to be Pap test negative have 5 percent chance to progress into disease (SIL) within one year; this risk is doubled next year.⁷⁴ In CAP Guidelines for Review of Pap Tests in the Context of Litigation or potential Litigation, the College of American pathologists (CAP) has declared that “The Pap test is a screening test that involves subjective interpretations by a cytotechnologist or pathologist of the thousands of cells that are present on a typical gynecological cytology specimen. Studies indicate that an irreducible false negative rate of approximately five percent. Although rescreening can reduce the false-negative rate, zero-error performance cannot currently be attained.”⁷⁶

Although the purpose of screening is not only to detect cervical cancers, but also to detect and remove high-grade lesions (thus to prevent potential progression to cervical carcinoma), the Pap test sensitivity for high-grade cervical intraepithelial neoplasia (CIN) is in the range of 70 to 80 percent. Almost a half of the cervical cancers diagnosed in the United States are in women who have never been screened, and an additional 10 percent of cancers occur in women who have not been screened within the past five years. It yields approximately 20 to 30 percent cancers occurring in women who have been falsely diagnosed as negative. False-negative results occur even in optimized screening programs and cannot be entirely eliminated.⁸⁶

Today, Pap test is a standard of health care for American women, which is provided in more than 3,000 cytopathology laboratories of laboratory units, by more than 10,000 professional, technical and administrative personnel. This infrastructure is responsible for 50 million tests per year, or a billion dollar/year health-care businesses.

In the United States, the Pap test is also one of the most regulated laboratory procedures. It is regulated by CLIA *88 (Clinical Laboratory Improvement Act), oversight by FDA (recent regulations) and CDC (Centers for Disease Control and Prevention), and is conducted according to numerous guidelines produced by professional societies and special interest groups. Research for improvement of Pap test (mostly improvement of accuracy) has been supported by NIH, and private industry (medical devices).

American experience with Pap test and the effects of this test on reduction of cervical cancer mortality rates (from more than 11 in 1950 to less than 3 in 1998)⁷⁴ was so impressive that this test has spread all over the world. According to WHO, in recent years, three out of four deaths from cervical cancer (still the major killer of women from malignant diseases) come from countries where Pap test is not available.⁷³

Unfortunately, this the most recognized cancer screening test, in spite of the whole success, has its own obstacles. In 1996, the NIH Consensus Conference on Cervical Cancer revealed that one out of five women diagnosed with cervical cancer had Pap test negative result within five years prior to the cancer occurrence.⁸⁹ This was an unacceptable rate of false negative diagnoses (20%) and the Conference called for improvement of the test. A magnificent effort was invested into this task. The most relevant achievements are summarized in the Prior Art section.

4.2 Prior Art

U.S. Government issues policy and regulations on Pap test improvement and designates its agencies (e.g., FDA, CDC, CMS) to enforce compliance of Pap test providers and facilitators; professional societies (e.g., ACS, CAP, ASCP, ASCT) issue guidelines and use licensing and accreditation for protecting this policy; the Pap test providers comply with the policy and provide feedback with inventions and suggestions, and the medical device industry produces new tools to assist providers to improve the quality of the Pap test. Certain aspects of this effort are directly related to our invention. This “prior art” is presented below; we have added only comments on how our invention is, or might be, connected.

4.2.1 Policy on Pap Test (Regulation and Guidelines)

a. Code of Federal Regulations (CFR), Title 42, Part 493.1275: Cytology, and other paragraphs related to diagnostic tests of moderate complexity. (www.access.gpo.gov/nara/cfr).⁵⁹

CPT is in development to become a new in vitro diagnostic medical device of moderate complexity. CPK is a tool to make it happen.

b. Centers for Medicare & Medicaid Services (CMS) regulates all laboratory testing (except research) performed on human in the U.S., through the Clinical Laboratory Improvement Amendments (CLIA). Pap test is regulated by the CLIA Regulations Part 493 (last revision Dec. 29, 2001) publicly available at the Centers For Disease Control (CDC) site.(www.cdc.gov/clia).⁸⁴

The act describes requirements for a laboratory to register for performing Pap test, certificate of accreditation, proficiency testing, patient test management, quality control, personnel requirements, quality assurance, inspection and enforcement procedures.

These regulations relate upon three standard classifications: CIN (cervical intraepithelial neoplasia) for histology,⁹³ TBS (The Bethesda System)⁸⁹ and 2001 Bethesda System for cytology.⁷⁸ In addition, CDC, Division of Laboratory Systems has developed standards for cytotechnologist/cytopathologists training and proficiency testing in form such as collection of slides (Laboratory Medicine, Cytology Proficiency Testing),⁸³ or digital images (Cytoview™).⁷⁵

All these standards are developed on microscopic images of cervical specimens stained by H&E (hematoxylin/eosin), or Papanicolaou (extended H&E)⁹³ staining techniques. Neither classifications nor any standard image has ever mentioned cervical acid phosphatase as a new biomarker for cervical cancer screening. However, the most recent American Cancer Society Guideline for Early Detection of Cervical Cancer (November 2002)⁸⁵ acknowledges the presence of a new technology in development which is intended to enhance the image of abnormal cells.⁸⁶ This description stands for CPT. Obviously, the lack of prior acknowledgment makes our invention unique and special.

c. Professional Societies

In 1998, an Intersociety Working Group for Cytotechnologies, comprising the American Society for Cytopathology, American Society for Clinical Pathology, American Society for Cytology, the College of American pathologist, and the International Academy for Cytology) issued the Guidelines for Primary Screening Instrument for Gynecologic Cytology.⁸⁷ This document recommended an adjudicated cytology ( consensus of independent pathologists) as a “gold standard” for evaluation of the performance of new devices for primary cervical cancer screening.⁸⁷ This controversial recommendation (subjective evaluation of screening devices) was reconfirmed in 2000 by the International Consensus Conference on the Fight Against Cervical Cancer (Organized by IAC, and cosponsored by: The American Society of Clinical Pathologists (ASCP) , The American Society for Colposcopy and Cervical Pathology (ASCCP), The College of American Pathologists(CAP), The American Society for Cytotechnology (ASCT), The Center for Disease Control and Prevention, The International Union Against Cancer (UICC), and many international societies.⁸⁸

d. In 2002, The College of American Pathologists (CAP) issued CAP Guidelines for Review of Pap Tests in the Context of Litigation or Potential litigation. In this document, CAP acknowledges that the subjective evaluation of Pap test has an irreducible 5% false negative rate.⁷⁶

e. NIH Consensus Conference, Bethesda 2001

In 2001, NIH organized a Consensus Conference on Terminology for Reporting Results of Cervical Cytology. The Conference definitely eliminated the term “diagnosis” from Pap test screening procedure, and replaced it with “interpretation” or “result” of Pap test. In addition, the new terminology adopted new names trying to emphasize the dichotomous nature of screening: negative/normal and positive/abnormal.⁷⁸ This approach is very encouraging for our invention, which result is also reported as Y/N acid phosphatase in squamous cells—yes, meaning positive/abnormal, and no, meaning negative/normal result.

f. ASCCP: 2001 Consensus Guidelines for the Management of Women With Cervical Cytological Abnormalities, provided evidence-based consensus guidelines for the management of women with cervical cytological abnormalities and cervical cancer precursors. Once again, these guidelines put emphasis on colposcopy with biopsy as the diagnostic measure necessary for evaluation of screening results.⁸²

All these and many derived guidelines made the Pap test one of the most regulated laboratory procedures in the U.S. Our inventions, presented in this application, are in compliance with the aforementioned regulations.

4.2.2 Opportunity to Save Lives (Public Needs)

Cervical cancer biology is characterized by slow progress and many pre-cancerous stages that might be recognized on cytological specimens and appropriate measures taken to prevent disease progress and save lives. The most recent nomenclature of the clinical conditions that can be found on cervical specimens (The 2001 Bethesda System)⁸¹ was immediately followed with 2001 Consensus Guidelines for the Management of Women with Cervical Cytological Abnormalities.⁸² Both documents are expected to have a major impact on the standard of health care for American women, and on the Pap test market (medical devices used for Pap test performance).

American Cancer Society Guidelines

The American Cancer Society (ACS), a professional society sponsoring the Pap test promotion from 1950 to present, recently revised their Guideline for Early Detection of Cervical Cancer. This document recognizes the value of new technologies for cervical cancer screening, and is suggesting new, something prolonged periods between regular Pap tests. It is another evidence that new, more accurate, and prognosis meaningful methods are welcome—it is exactly what CPT is bringing. This Guideline has mentioned our technology under the category of new, in development, technologies intended to enhance the visibility of abnormal specimens.^{86,89}

4.2.3 Improvement of Sampling and Distinction Between the New Devices and the Proposed Invention(s).

The 1996 NIH Consensus Conference on Cervical Cancer revealed an unacceptable rate of 20% false negative results.⁸⁹ Many researchers, policymakers, federal institutions and professional societies have addressed this issue. A consensus exists that the main cause is an error. Differences are about what error is primary and how to reduce this error. In summary the following errors are most frequently cited:

Not having a Pap test within 5 or 3 years before disease progress.

Sampling error.

Technical error (specimen preparation and/or staining).

Interpretation error.

Not compliance with recommendations.

The medical device industry has provided many devices to improve the technical aspects of Pap test. The most related to our invention are listed below.

4.2.3.1 Sampling Devices

a) Medscand Medical⁶¹ (www.medscand.se)

Cytobrush Plus®

Endorette®

Plastic spatula

These devices could be used for sampling quality specimens for CPT.

b) Cytyc, Corp.⁶² (www.thinprep.com)

Cervical Broom

Filters for ThinPrep Processor

Coated slides for ThinPrep®Pap Test™

CPT does not need specimen filtering and it does not utilize filters or ThinPrep Processor. However, according to our experience, using filtration is providing very nice and clean microscopic images that appeal to pathologists more than the recommended technique (monolayer).

4.2.3.2 Spray Fixatives

Air-drying of specimens was considered as an important cause for morphologic artifacts.^{79,93} Several companies have addressed this problem with ethanol-based fixatives sprayed on Pap smears immediately after preparation.

Cytoprep Fixative (Fisher)

Spray-Cyte (Becton, Dickinson)

Cytology Fixative (Surgipath)

Sprayfix Aerosol (Surgipath)

Carbowax (Polyethylene Glycol)

The presence of ethanol is detrimental for acid phosphatase activity. We are working on a spray fixative based on methanol. Currently, we use methanol-acetone-formaldehyde solution. Acetone and methanol are product of Fisher Sci., and we use low formalin concentration (4% Formaldehyde, Medical Chemical Corp. Torrance, Calif.).

We have also seen that hydration procedure may improve nuclear staining. However, better hematoxylin is needed. A double strength hematoxylin is provided by Ricca Chemicals (Arlington, Tex.).⁶⁷

4.2.3.3 Collecting Solutions

a. ThinPrep® PreservCyt® (Cytyc, Corp)⁶¹

PreservCyt is the only cell preservative solution based on methanol (U.S. Pat. No. 5,256,571).⁷ Having known that acid phosphatase inhibition is methanol concentration dependent^{96,97,101} and knowing that PreservCyt® contains 50% methanol, we tested and identified this solution as compatible with CAP-PAP test. Since then, we have been using PreservCyt as a standard for comparison with our own solution CAP-PAP Test Solution (CPS).⁵⁶

PresrvCyt does not protect cervical acid phosphatase activity for more than 1-2 weeks. Therefore, the enzyme protecting solution had to be introduced. CPS is considered to be this type of cell-enzyme-protective solutions.⁵⁶

b. SurePath (TriPath Imaging)⁶³ (www.tripathimaging.com)

SurePath™ Test Pack is a cell-preservative solution based on ethanol (<24%), which was intended for cervical specimen collection, storage (4 weeks at RT), and transport on microscopic slides via PrepStain™ Slide Processor.

This is an excellent system, but it is not compatible with our system because of ethanol that inhibits acid phosphatase.

c. ThermoShandon⁶⁴ (www.thermoshandon.com)

Cyto Rich Red® Collection Fluid. Ethyl alcohol based.

Cytospin Collection Fluid. Based on alcohol/carbawax (ethyl alcohol and polypropylene glycol).

Cell-Fixx carbawax-based

These solutions are incompatible with acid phosphatase in our test.

d. Surgipath Medical Industries⁶⁵ (www.surgipath.com)

Cyto Jar® is a solution for convenient collection, fixation and preservation of all cytological specimens. The preservative contains polyethylene glycol. For Pap staining, this alcohol is slowly removed with ethanol.

Sed-Fix® is ethanol-based collection and preservation solution to be used with the Cyto-Sed® specimen preparation device.

This solution is not compatible for CPT.

4.2.3.4 Transferring Devices

a. ThermoShandon line of products: Cytospin 2 centrifuge with disposables (Cytofunnels, Megafunnels, Cytoclips, Coated slides).⁶⁴ (www.thermoshandon.com).

We are using this system for transferring specimens from CPS suspension onto microscopic slides. The result is a round (cytofunnel) or quadrangular (megafunnel) thin-layer of cells.(FIG. 10) Multiple technical problems include, but are not limited to, cell clumping, detritus, obscuring cervical cells with inflammatory cells, and “rim” distribution. It is necessary to synchronize the cell density, spinning time and speed in order to obtain an evenly distributed monolayer of cervical cells.

b. Cytyc Corp.'s ThinPrep® Processor 2000(3000)62 (www.thinpre.com)

The tabletop ThinPrep 2000 and the fully automatic ThinPrep 3000, are processors for transferring cervical specimens from the specimen collection fluid (PreservCyt®) onto ThinPrep microscopic slides before staining. The system is based upon a patented filtering process. Suspension of cells is filtered—all small components of this suspension (debris, inflammatory cells, mucus) are either destroyed or passed throughout, cells are retained on the filter and than transferred onto microscopic slides. The result is a round thin-layer of cells free (more precisely reduced) from debris, inflammatory cells, and smaller cervical cells (including endocervical). (FIG. 10) Microscopic image is reasonably clean and much more appealing to pathologists tired of Pap smears (inadequate Pap smear contain lot of vaginal fluids, mucus, inflammatory and exfoliated cells; adequate smear should contain only abraded cells—but, this is not usually the case).

The major disadvantage of this technology is that many small and large abnormal cells can be missed (passed through the filter, retained in the container, or destroyed in the collecting solution); thus, false negative rate could be the same or higher than with the Pap smear. Other disadvantage is the cost: filtering technology is much more expensive than centrifugation (Cytospin) or cell sedimentation (Cyto-Sed).

The major advantage is that the specimen is retained in the solution for a week or two after slide preparation, and additional slide may be made of the residual material instead of the new sampling.

We have used ThinPrep Pap Test (PreservCyt suspension and ThinPrep 2000) as a standard control for our study on CPS.⁷

c. Surgipath Medical Industries (New Device: Cyto-Sed)⁶⁵ (www.surgipath.com)

Recently, this company has introduced Cyto-Sed™ cell transferring device. It was approved for other cytological specimens, but not for cervical. We are evaluating this device and, in spite of insufficiencies, we think it can be effectively used in low-cost settings.

4.2.4

Improvement of Staining and Distinction Between the New Devices and the Proposed Invention(s).

4.2.4.1 Sigma Chem. Co. (St. Lois, Mo.)⁶⁶ (www.sial.com)

Sigma is a well-established provider of biochemicals and reagents for life science research (www.sigma-aldrich.com)

Sigma is one of the major providers of Papanicolaou stains (OG-6, modified EA, EA-50, Hematoxylin, Scott's tab water, reagent alcohol) and reagent kits for acid phosphatase (Acid Phosphatase Leukocyte kit No.387A and #386A, and Acid Phosphatase Lymphocyte kit No. 81A), individual reagents for those kits, cytology/histology fixatives, and mounting media. All cited reagents are approved for in vitro diagnostic use.

None of these kits has ever been used for cervical acid phosphatase. We are using Sigma individual chemicals for preparation of reagents included in CPK (acid phosphatase substrate, diazonium salt, components for buffers, sodium nitrite). Papanicolaou stains are well accepted and used worldwide. Sigma's glycerol-gelatin mounting medium is used in CPK.

4.2.4.2 Ricca Chemicals (Arlington, Tex.)⁶⁷ (www.riccachemical.com)

Ricca manufactures Papanicolaou stains including Hematoxylin (Gill 3 triple strength), OG-6, and EA 65, and EA-5, cytology/histology fixatives and reagent alcohols.

We have found that Ricca's Gill 3 triple strength hematoxylin provides fast and transparent nuclear staining, that is appropriate for CAP-PAP test. Hematoxylin Gill 3 contains hematoxylin, sodium iodate and aluminum sulfate.

4.2.4.3 Surgipath Medical Industries (Richmond, Ill.)⁶⁵(www.surgipath.com)

Manufactures and sales Papanicolaou stains: Hematoxylin, OG-6, EA-65, and EA-50, and reagent alcohols, xylene and cytology/histology fixatives.

We have found that Surgipath's OG-6 and EA-65 are compatible with CAP-PAP test.

4.2.4.4 ThermoShandon (Pittsburgh, Pa.).⁶⁴ (www.thermoshandon.com)

Manufactures and sales Papanicolaou stains including Hematoxylin, EA-65, and OG-6, alcohols, and xylene.

We have not found these stains to be the best choice for CPT.

4.2.4.5 Richard-Allen Scientific (Kalamazoo, Mich.)⁷² is a subsidiary of Apogent Technologies (www.apogent.com)

RAS manufactures and sales Papanicolaou stains including hematoxylin, OG-6, EA-50, EA-65, clarifiers (alcohols), xylene, cytology/histology fixatives, and mounting media.

We have found that these reagents are not a good choice for CPT.

4.2.4.6 Fisher Scientific⁶⁸ (www.fishersci.com)

Fisher Scientific manufactures and sales Papanicolaou stains, Hematoxylin, EA-65, EA-50, OG-6, alcohols, cytology/histology fixatives and mounting media.

We are using Fisher-brand methanol, alcohols, and acetone.

4.2.5 Human Papilloma Virus Issues

4.2.5.1 HPV vaccine⁶⁹(www.vanderbilt.edu/reporter)

HPV is a sexually transmitted disease that infects 50 percent of the population at some point. There are more than 90 types of HPV, but four of the identified types are responsible for most cases of genital warts and 70 percent of cervical cancer. Vaccines against HPV infection are in development. In 2002/2003 the first clinical trials (Phase III) have been initiated worldwide. However, since the endpoint is NOT occurrence of cervical cancer in protected women, there will be more than 10 years before any significant conclusion be made. The most serious dilemma regarding further success of these vaccines was raised by Garnett and Waddell in 2000.⁹⁸ Referring to cervical cancer screening as a well established and efficacious preventive measure based on cervical cell morphology, they asked whether anti-HPV vaccination will alter biology of cervical epithelium and ruin the value of cytological diagnosis. If this is going to be the case, than risks of vaccination will overcome the potential benefit. An answer to this question has not been published yet.

4.2.5.2 In Vitro Diagnostics

Digene Corp., Gaithersburg, Md., (www.digene.com) has introduced the Hybrid Capture® 2 HPV DNA Test for detecting human papilloma virus in cervical-specimen collecting solutions. FDA has approved this test for providing additional information in equivocal cases that receive mildly abnormal or ASC-US Pap results. The company's target is 3M tests per year.

The problem with this technology is its cost vs. benefit ratio. At present, the HPV DNA test is performed only on residual suspensions of cervical, cells let after the ThinPrep Pap Test is completed. According to the American Society for Colposcopy and Cervical Pathology, the 2001 Consensus Guidelines on Management of Women with Cytological Abnormalities⁸² is recommending repeat Pap test, colposcopy and HPV DNA testing. However, although 31%-60% of women with ASC-cytological diagnosis test positive for HPV, it is not known how to proceed with them, and colposcopy is preferable. This approach poses questions: “Why testing HPV? Why not recommending colposcopy directly?”

In our studies, we were able to see cervical acid phosphatase positivity in HPV infected cells (FIG. 5, lower-left). The value of this information is under consideration. HPV testing is compatible with our solution (CPS). HeLa cell line is heavily loaded with HPV, and highly positive (score about 300 out of 400 possible) for cervical acid phosphatase. However, Pap test (and CPT) is anti-cancer screening test, not a HPV screening test. Surgery is therapy of choice for cancer, not for HPV. Cervical cancer is not infective disease and vaccine will never help once cancer evolved. Therefore, we believe, the quest for more accurate methods for diagnosis of cervical cell abnormalities signaling precancerous conditions will continue, at least, for the next ten years. I.

4.2.6 Improvement of Interpretation and Reporting the Pap Test and Distinction Between the New Devices and the Proposed Invention(s).

In 1996, an NIH Consensus Conference on Cervical Cancer revealed that 20% (one out of five) cervical cancer patients had had negative Pap test reading within the recent five years.⁸⁹ Among other recommendations to improve this unacceptable high false negative rate, the Conference proposed an inclusive cytology classification (The 1991 Bethesda System, or TBS) to standardize cytological reporting of the Pap test. TBS was widely accepted and became a standard of health care in the U.S. However, early enough, it was recognized that distinction between normal and abnormal specimens was not clear. The threshold was in the midst of a category that was defined as ASCUS (abnormal squamous cells of undetermined significance). In 2001, another NIH Consensus Conference was held to improve the first classification. The new cytological classification for reporting Pap test results (Bethesda System 2001) is now in effect.⁸¹ The Consensus Statement on Terminology for Reporting Results of Cervical Cytology⁷⁸ clarified that cervical cytology is only a screening method; consequently, the term “diagnosis” has been replaced by “interpretation” or “result” to convey that cervical cytology provides an interpretation of morphological findings that must be integrated into a clinical context. The classification is simplified. Specimens for which no epithelial abnormality is identified are reported as “negative for intraepithelial lesion or malignancy” or NIL. This category includes old categories WNL and BCC. The Conference defined specimens with Squamous Intraepithelial Lesion (SIL) but, again, left open the ASCUS category. Instead of ASCUS, two new categories were introduced: ASC-US (atypical squamous cells of undetermined significance) and ASC-H (hoped that ASC-H has a positive predictive value for histological CIN 2 and 3).⁷⁸

According to the American Cancer Society data (2002) approximately 50 million women are screened yearly, and approximately 7.1% (3.6 million) receive an abnormal result (two million ASC-US and ASC-II, 1.25 million LSIL, 300,000 HSIL and 13,000 invasive cancer).⁸⁶ Obviously, the consensus conferences have created a usable reference for epidemiological studies and for a policy targeting the Pap test improvement. For instance, HPV DNA testing is targeting ASC-US population to select those who might have been missed with cytology alone.

In spite off all improvements, the College of American Pathologists has issued a Policy on Guidelines for Review of pap tests in the Context of Litigation or Potential Litigation. In this document they wrote: “The Pap test is a screening test that involves subjective interpretations by a cytotechnologist or pathologist of the thousands of cells that are present on a typical gynecologic cytology specimen. Studies indicate an irreducible false negative rate of approximately 5%.⁷⁶ Although rescreening can reduce the false negative rate, zero-error performance cannot currently be attained. Many factors, including the subjectivity involved in interpreting difficult cases and sampling problems with specimen collection, prevent zero-error performance.”⁷⁶

BioSciCon, introducing a novel biomarker for cervical cell abnormality, challenges this 5% irreducible rate by enhancing the visibility of abnormal cells and by increasing the awareness of the examiners that abnormal cells are present.^{54,55}

4.2.7 Automation

Varistain Gemini Automatic Slide Stainer (ThermoShandon) was designed with internal heated station to dry slides. We have asked ThermoShandon to customize this instrument with a wet (instead a dry) heated station. They built a thermo-chamber, put it under computer control, and Gemini became the first in the world automatic slide stainer capable for fully automatic processing of methods requiring enzyme reaction at 37° C. for up to one hour. Application of this improved Gemini for CPT staining is described in section 8.2.2 (and 9.2.2, CPAS, Alternative Embodiments).⁹⁰

4.2.8 Speculoscopy (www.papsure.com)

Recently, the FDA has approved a speculoscopy as the first in-office method for improving sensitivity of Pap test. This method uses white vinegar (5% acidum aceticum) to enhance visibility of epithelial lesions on cervix uteri, and an optical instrument with blue light for through-speculum examination of the cervix. It is similar to colposcopy and carries a certain degree of discomfort, but without consecutive biopsy it seems to be an unnecessary addition between Pap test and colposcopy.

The name of this method is PapSure® and is sponsored by Watson Diagnostics, Inc. (subsidiary of Watson Pharmaceuticals, Inc., Corona, Calif.).⁷¹ (www.papsure.com)

4.3 History of the Invention

4.3.1 BioSciCon's Novel Approach

In 1986-88, we were examining whether the image analyzing systems could improve clinical pathology diagnostic procedures. We have tested a number of cell specimens versus a list of cytochemical reactions (known to be of a certain clinical value) and we used image analysis to measure the success of matching. One of the surprises was the match between cervical cells (Pap smear) and acid phosphatase. Unexpectedly, we detected acid phosphatase positivity among abnormal squamous cervical cells in cases already identified as Pap test positive.³ Much later, in 1996, when improvement of Pap test became a hot issue, we returned to this old information, we found that it was exclusive, and we decided to develop a test that will integrate detection of acid phosphatase inside abnormal cervical cells and Papanicolaou staining as background for cytological diagnosis.^{1,2} Our efforts were rewarded, and on Nov. 7, 2000, we received approval for our patent “CAP-PAP Test.”¹

After completing preclinical studies, we have started clinical laboratory trials supported by U.S. Government via SBIR-NIH-NCI grants. In these trials (completed, ongoing or planned) we have used commercially available reagents for our test. During this period, we have realized that a kit will be more productive, easier to use, less expensive, and better controlled.⁷ This development (following the parent patent) has produced a series of new inventions. Consequently, we are proposing the most relevant of them for patent protection. The following is a brief description of the important milestones we have achieved in this process and the inventions that have occurred along.

Method (Service, Assay, Procedure)

We have not invented any of dyes or staining materials described above. Most of them had to be slightly modified to meet the requirements of the new indication.

However, we have invented a method to visualize cervical acid phosphatase inside abnormal squamous cells on cervical specimens counterstained with a modified Papanicolaou staining technique, and we were first to recognize the potential of this method for cervical cancer screening.⁵⁵

CPT (CAP-PAP Test)¹(http://pathf.uspto.gov/netahtml/srchnum.htm)

Our method, as described in CPT patent (U.S. Pat. No. 6,143,512) includes:

Preparation of cervical specimen as a smear or a monolayer on microscopic slide.

Hydration/Fixation of the specimen (methanol-acetone-formaldehyde).

Marker processing/enzyme visualization (the exposure of cervical cells to an acid phosphatase specific substrate, (substituted naphthyl phosphate) at 37° C.; intracellular acid phosphatase liberates naphthol from the substrate that is simultaneously coupled with a diazonium salt producing a brilliant red color pigment (insoluble enzyme product deposit) that precipitates on the sites of enzyme activity. We are using Sigma individual chemicals as a basis for preparation of reagents included in CPK (acid phosphatase substrate, diazonium salt, components for buffers, sodium nitrite).

Cytological staining (staining of cellular morphological details) using a modified Papanicolaou technique. Hematoxylin with bluing reaction for staining the nuclei, Orange G and Eosin alcohol-based red stain (EA-65) for counterstaining of the cytoplasm.

Mounting slides with water-based mounting medium

(glycerol gelatin).

Interpretation of results

A brilliant red pigment (the marker) is clearly distinguishable on the bluish background of cytology specimen. Probability to miss labeled cells are much less (0.01) than without the marker (0.10).(CPT pat ) (See FIG. 2, FIG. 3, and FIG. 5) Normal squamous cells do not contain acid phosphatase. Therefore, presence of the marker indicates abnormality and requires careful examination and interpretation. Absence of the marker indicates to a high probability for the lack of abnormality.

We have also invented TOOLS to assist method provider to perform the service more accurately, faster, better controlled, less expensive and easier than with other commercially available tools.

Tools (Product, Device)

CPK (CAP-PAP Test Kit)⁹

CPK is an assembly of reagents, procedures and controls put together in a kit to enable laboratories to perform CPT more accurately, more consistently, faster and better controlled (both QC and QA).

CPS (CAP-PAP Test Solution)⁷

CPS makes CPT applicable to cervical specimens collected in solution.

CPAS (CAP-PAP Test Automation Software)⁸

CPAS indicates that automation could only improve CPT.

All three products (CPK, CPS and CPAS) have been conceived, designed and developed as prototypes during the CPT research and as an integral part of this research.⁵⁵⁻⁵⁷

The following events were the milestones for development of our method from an idea throughout prototype and clinical trials towards an in vitro diagnostics medical device.

4.3.2 CAP-PAP Test Patent (CPT)¹

U.S. Pat. No. 6,143,512; granted on Nov. 7, 2000.

From the Abstract:

The CAP-PAP Test is a double-staining, single-slide, microscopic method. An in vitro diagnostic medical device for manual and automatic staining and interpreting of the Pap smear for purpose of cervical cancer screening, diagnosis of cervical dysplasia and follow-up of therapy can be developed using this double-staining, single-slide microscopic method. Abnormal cervical cells are labeled with an intracellular acid phosphatase derived pigment (azo-dye) to improve visibility of abnormal cervical cells on conventionally stained Pap smears. The enzyme marker improves human perception and/or sensitivity of automatic instruments when distinguishing cell abnormality and interpretation of Pap smears. Increased accuracy of CAP-PAP vs. Pap test is expected to reduce false negative readings of the conventional Pap test. A rapid manual version of the test is low cost, does not require additional personnel training and is instantly applicable in all cytopathology laboratories is provided. The invention further provides a diagnostic kit, an automatic stainer and an evaluation device for performing the double staining-single-slide microscopic method.

We have applied our method and the kit in a routine practice of two cytopathology laboratories serving as contract research organizations for laboratory services required for BioSciCon's clinical trials (assessment of safety and efficacy of the new method in comparison with the standard). Most of the examples, presented further in this application, have been produced independently.

4.3.3

CAP-PAP Test for Cervical Cancer Screening (Clinical Laboratory Trials SBIR-NIH-NCI)

4.3.3.1

NIH Grant # 1 R43 CA86767-01: CAP-PAP Test for Cervical Cancer Screening.⁵⁴

Objective:

To study whether the CAP-PAP test (CPT) could increase cytotechnologists' perception of cytological abnormality on Pap smears to a level that a cytotechnologist using CPT could reduce the false negative rate of the primary cervical cancer screening based on the Pap staining alone.

Study Design:

Multi-center, random selection, assessors blinded, clinical trial using a split-sample, two paired-group design to study sensitivity of cytechnologists using CPT versus sensitivity of cytotechnologists using conventional Pap staining to detect disease (Bethesda System for Reporting Cervical/Vaginal Diagnoses) during primary screening of Pap smears obtained from women-at-low risk for cervical cancer coming to doctor's office for regular Pap check-ups.

Results showed that CPT assisted cytotechnologists improving their screening ability in comparison with a historical group (Pap test). In a 2-independent-group design, using an adjudicated cytology standard applied at rescreen and review (quality control) the following results were obtained: CPT/Pap sensitivity=0.93/0.67; specificity 0.96/0.85; PPV=0.33/0/21; NPP=0.97/0.90. These results justified our application for an SBIR Phase-2 grant that was awarded consequently.^{54,55}

4.3.3.2

NIH-NCI Grant # 2 R44 CA 86767-02: CAP-PAP Test for Cervical Cancer Screening 2.⁵⁵

Objective:

1. Same as in Phase-1.

2. To extend the follow-up period for two years per a subject (or three years for the study) in order to add a clinical outcome standard (disease occurrence/progress) to the adjudicated cytology standard (used in the Phase-1, and to increase the study sample size up to 1,800 subjects to preserve statistical power.

3. To acquire preliminary information, through two pilot informational efficacy studies, on prospective for (1) CPT application on liquid-based specimen collection technology, and (2) CPT options for automation of the marker processing procedure.

This is an ongoing study entering the last quarter for recruitment. The most recent study assignment number was 1,500. We conduct regular monitoring of six clinical sites and two laboratory cites, and collect information from case report forms for our central database. Forms are included in the Appendix.⁹⁹ Although the study is blinded, and we cannot compare paired groups, due to an evidence-based model, we are able to monitor decisions made on primary screening, rescreen and review level for CPT and to compare with Pap test reports (only summaries).⁹¹ (FIGS. 6 and 7 with FIGS. 2, 3 and 5) In general, we have seen monthly repetition of the same trend: The primary screener identifies more abnormal slides (˜20% vs. 10%) for review, and makes less omissions (false negative rate 1% vs. 10%); reviewer identifies twice abnormal slides more than using Pap test only (˜10% vs. ˜5%). Another important observation is that until now, no one normal-looking squamous cell was positive (among hundreds of thousands) and not a single abnormal-looking squamous cell was CAP negative (among thousands). This study continues.^{91,99}

4.3.4 CAP-PAP Test Kit

Reference:

1. Invention Disclosure: U.S. Pat. No. 498,142 of Jul. 31, 2001.⁹

2. Provisional Patent: U.S. Ser. No. 60/348,514 of Jan. 16, 2002.⁹

In the beginning of clinical laboratory trials (1999), we were supplying laboratories with standard reagents (from other manufacturers) and our instructions how to use these reagents to perform CPT. Very early we realized that reagents, available commercially, are not sufficiently consistent or stable to be safely used in our research. Therefore, we decided to assemble enough reagents for a period of approximately six months and to use only this batch. This was an expensive idea and soon, we decided to buy raw material and prepare our own reagents. It was more affordable. Further, we have realized that by modifying reagent composition we may improve the outcome and to optimize the method for the best presentation of cervical acid phosphatase on CPT smears. Some of these improvements qualified for inventions and we decided to start the quest for development of our own kit. Therefore, the idea to produce a kit came as surprise for us; it was a new quality in our thinking after we found ourselves unsatisfied with the available reagents. This new quality needed a certain amount of research, development of prototypes, testing, etc., before we came to the current point.

In 2001, we completed and tested a prototype (FIGS. 8 and 12). We also tested short-term stability of reagents. The long-term stability of reagents and efficacy of the kit to enable better performance of CPT in comparison with individual reagents will be tested in a new study (submitted for another SBIR Phase-2 funding).⁵⁵

The major novelty in this development, in addition to providing standardized means for testing acid phosphatase, was the introduction of COMBO control slides for QC/QA.

(For details see the Main Embodiment Description and Operation)

4.3.5 CAP-PAP Test Solution

Reference:

A. Invention Disclosure: U.S. Pat. No. 487,457⁵

B. Provisional Patent: U.S. Ser. No. 60/271,480 of Feb. 27, 2001)⁷

Our study BSC-0002 “CAP-PAP Test for ThinPrep Specimens” that was recently completed,⁹⁴ clearly demonstrated that CPT can improve the visibility of abnormal cells on ThinPrep slides, even to improve visibility of endocervical cells—identification of endocervical cells is an recognized problem for ThinPrep slides interpretation. (FIGS. 2C and 2D) Three additional problems we incurred were: (1) rapid disintegration of cells suspended in PreservCyt solution with creation of debris, (2) high cost of ThinPrep Processors for filtering this debris, and (3) rapid reduction of cervical acid phosphatase activity after two weeks.

At the same time, we realized that manufacturers of devices for transferring cells from suspension onto microscopic slides (such as ThermoShandon) have encountered the same problems, but, to overcome the problem of debris, they have being developing their solutions (e.g., ThermoShandon: Papspin). As many other companies, we decided to develop our own solution with purpose to protect cell enzyme and morphology equally, and to use less expensive cell-transferring device(s).

After a series of preclinical studies, in 2000/2001 we conceived a working formulation of a solution that should protect enzyme activity as well as cell morphology for at least the same duration of time as the best cell-preservative solutions on the Pap test market. This new formulation was tested in two studies (1 R43 CA 94628-01), efficacy was confirmed, and we applied for an SBIR Phase-2 grant in order to test the efficacy and safety of this formulation in clinical environments.^{56,95}

The major invention that happened during this endeavor was an incidental addition of ICM (novel antioxidant, U.S. Pat. No. 5,620,961)⁵⁷ a fructose ester with a short fatty acid that, seemingly, improved the cell protective function of our solution. For details, please see the Alternative Embodiments Description and Operation.

4.3.6 Automatic Slide Staining⁹⁰

There are many automatic slide stainers on the market. ThermoShandon is among the leading manufacturers of these devices. In our research we are using several devices from this manufacturer, including Cytospin-2 centrifuge and disposables (for cell transfer). This manufacturer built for us a prototype of an automatic slide stainer with wet heated station (for temperature controlled enzyme reaction in solution). It is an advanced version of their best instrument of this kind Varistain Gemini Automatic Slide Stainer (www.thermoshandon.com). We have used this instrument for CPT marker processing and cytology staining procedure, we confirmed its feasibility for our purpose, and we developed an algorithm for use of this instrument in the new indication. Because our test is a novelty, this instrument is a novelty; we feel that our algorithm is a novelty, too. For details, please see the Alternative Embodiments section Description and Operation.

4.3.7 ICM⁵⁷

ICM is an acronym for Inclusion Complex Molecule comprising of a fructose-hexanoate ester included inside β-cyclodextrin. This is a new molecular entity described in our previous patent (10, U.S. Pat. No. 5,620,961).¹⁰

The patent claims that this molecule entering cells donates energy (ATP) and hydrogen ion, and that via NAD/H and NADP/H mechanism, restores the reduced glutathione, thus, improves cell defense mechanism against oxidant radicals damage.

That patent described the use of ICM for prevention of doxorubicin-induced cardiotoxicity. Here, we are describing another use of ICM: cell-preservation in liquid media.¹⁰

For details, please see the Alternative Embodiments Description and Operation.

4.3.8 ImmunoCAP⁶

In 2000, we initiated research for immunohistochemical visualization of CAP. The idea was to isolate HeLa cell line acid phosphatase and using hybridoma technology to produce antibody against acid phosphatase protein; to label this antibody with peroxidase, and to visualize antigen/antibody reaction with avidin-biotin complex and consequent color development with 3,3 diaminobenzidine-HCl, and hydrogen peroxide. We have left this project for the future when thechange of the current technology will become unavoidable.⁶

5.0 Competitive Advantages

Better Accuracy

Current data (Nov. 2, 2002, report)⁹¹ suggest that CPT has sensitivity above 91%, specificity 97%, positive predictive value of 76% and negative predictive value of 99%. It is much better than the conventional Pap test or ThinPrep Pap test (using adjudicated cytology as gold standard). CPK is adding to this sensitivity because it is making the marker better visible (sharply and distinctly localized) than the non-kit reagents.

Low Cost

Current sale price of CPK is estimated to be $360,00 per kit (300 tests), and $450.00 per box with solution vials.⁹²

Better Productivity

Time for primary screening a CPT slide is at average 3 min (due to the presence of the marker), and for rescreen (second screening of negative/normal slides) is 1 min. This speed, allows 100% negative slides to be re-screened; much better that the current standard of care requires (10% random negative and all high risk).⁹²

Less Liability

Introduction of control COMBO slides (see Alternative Embodiments) provides objective means for quality control and quality assurance for the first time in the history of the Pap test. Laboratories have opportunity for regular control of their performance (CPT marker processing and cytology staining) and a certificate of a continuous compliance with standards will reduce the legal responsibility of laboratories for false Pap test results.⁷⁶

### 6.0 BRIEF SUMMARY OF THE INVENTION

This invention discloses new products related to a unique patented process, and the use of these products to facilitate the process procedures.

The patented process is the Cervical Acid Phosphatase-Papanicolaou Test (CAP-PAP Test), or CPT.¹

A. The PRODUCTS

CAP-PAP Test Kit, or CPK⁹

CPK is an assembly of reagents, controls and instructions, assembled and organized in an operational unit to facilitate performing CAP-PAP Test faster and with better consistency.⁹ (FIGS. 8 and 9)

The kit is a novelty by itself:

It is a unique tool for performing CPT that, alone, is a novel test (method, service);

The kit includes novel products described below.

COMBO Control Slides or CCS⁵⁶

CCS is a set of microscopic slides included in the kit to serve for quality control and quality assurance of CPT and/or CPK. COMBO slides contain a standardized mixture of cells that are sensitive to acid phosphatase (HeLa cell line cells) and cytological staining (buccal cells). COMBO slides simulate abnormal cervical specimens. This product and its use for QC/QA is a novel concept never before used for evaluation or control of the Pap test results. (FIGS. 1 and 8)

CAP-PAP Test Solution, or CPS⁷

CPS is a solution intended for collecting specimens from cervical sampling devices, and for transporting, storage and transferring cervical cells from suspension onto microscopic slides. The novelty of CPS is that it can preserve both the enzyme activity and the cell morphology. Other, commercially available solutions are made only to preserve cell morphology. This new property of CPS was achieved with a novel formulation containing less alcohol and an antioxidant. An accidental adding of this antioxidant to the solution produced unexpected effects that we exploited later. (CPS PPA) (FIGS. 2E and 2F, FIGS. 3 and 9)

CPT Criteria for Reporting Results of Screening (CRRS)⁵⁷

CRRS is a set of instructions (with images) for reading CPT slides and for reporting results in clinically relevant terminology. CRRS is based upon the standard the cytological nomenclature (2001 Bethesda System), but it is modified to include cervical acid phosphatase as a new cytological marker of squamous cell abnormality.

CAP-PAP Test Automatic Staining (CPAS)⁸

SPAS is a software upgrade that combines our test (CPT), an automatic stainer (ThermoShandon Varistain Gemini) customized to meet requirements of CPT (wet heated station), and our algorithm that made this combination to work as a tool for automation of CPT. Both, the customized instrument and the software are novelties. (CPP-SAS ID)

Other proprietary products:

a. Labeling Insert: The Instructions (copyrights will be sought)

b. Kit assembly—Model (design patent will be sought).

c. Individual reagents (only when novelty is obvious, e.g., COMBO slides).

B. The USE

The above described products and the processes are intended for:

Demonstration/visualization of cervical acid phosphatase;

Detection of abnormal cervical cells obtained from abnormal cervical epithelium and smeared on microscopic slides either directly (from sampling device), or first collected in transporting solution and then transferred onto microscopic slides.

In particular, the novelty here relates to the USE of this technology (products and processes involved) for cervical cancer screening purposes.(14)

### FIGURES

FIG. 1.

MARK-PAP™ Test Kit Applied on Combo Control Slides

Cells collected in MARK-PAP™ solution.

CAP negative buccal cells and CAP highly positive HeLa cell line cells.

Microscopic magnification ×100, except upper right picture with ×40 magnification.

FIG. 2.

MARK-PAP™ Test Kit Applied on Different Cervical Cell Specimens

2-A and 2-B: MARK-PAP™ test kit applied on conventional Pap smears.

2-A: Three abnormal CAP positive squamous cells with three overlapping normally looking cells.

2-B: CAP positive abnormal squamous cell, inflammatory cells, and one CAP negative normally looking cell.

2-C and 2-D: MARK-PAP™ applied on specimens collected in ThinPrep solution.

2-C: CAP positive abnormal squamous cell.

2-D: Centrally positioned abnormal CAP positive cell.

2-E and 2-F: MARK-PAP™ applied on specimens collected in MARK-PAP™ solution.

2-E: Combo control slide. Centrally positioned CAP negative buccal cells surrounded with CAP positive HeLa cell line cells.

2-F: CAP squamous normally looking cells, two abnormal cells CAP positive, few inflammatory cells.

Microscopic magnification ×100.

FIG. 3.

MARK-PAP™ Test Kit Applied on Cervical Specimens Collected in MARK-PAP™ Solution

3-A and 3-B: CAP negative normally looking squamous epithelial cells. Microscopic magnification ×100 (3-1) and ×40 (3-2).

3-C and 3-D: Normally looking CAP negative and abnormal CAP positive cells.

3-C: microscopic magnification ×110. Print 3-D depicts, the same two cells on microscopic magnification ×40, with more CAP negative cells, and inflammatory cells.

3-E and 3-F: Normally looking CAP negative and abnormal CAP positive cells.

3-E: Three normally looking CAP negative cells, one endocervical CAP positive and one abnormal cell CAP positive cell, inflammatory cells. Microscopic magnification ×40.

3-F: Same cells can be seen in the center of the field on microscopic magnification ×20. More normal CAP negative cells and inflammatory cells.

FIG. 4.

Automatic and Manual MARK-PAP™ Test Applied on Control Combo Slides

Cells collected in MARK-PAP™ solution.

Upper figure: Automated MARK-PAP™ . One CAP negative buccal cell, four HeLa CAP positive cells.

Lower figure: Manual MARK-PAP™. One CAP negative buccal cell surrounded with five CAP positive HeLa cells.

Microscopic magnification ×100.

FIG. 5

MARK-PAP™ Test Kit Applied on Normal and Abnormal Cervical Smears

Upper left: Normal smear. CAP negative normally looking squamous epithelial cells.

Middle left: Two abnormal CAP positive cells ( LSIL), and normally looking negative cells.

Lower left: LSIL, abnormal HPV infected cells, CAP positive.

Upper right: Abnormal cells, CAP positive, HSIL.

Middle right: Abnormal cells, CAP positive, HSIL. Inflammatory cells, negative PMN, and two positive monocytes.

Lower right: HSIL, same group of cells as on upper right, higher microscopic magnification.

Microscopic magnification ×40, except lower two prints where the magnification is ×100.

FIG. 6

Cytology Tutorial on Cytologic Criteria for Pap Smears

Upper figure: Cytologic features of cervical dysplasia

Lower figure: Cytologic features of cervical squamous cell carcinoma http://medlib.med.utah.edu

FIG. 7

Conventional Papanicolaou staining

Upper figure: LSIL

Lower figure: HSIL

FIG. 8

MARK-PAP™ Test Kit

Individual regents and control slides.

FIG. 9

MARK-PAP™ Cytopreservative Solution

Accessories to MARK-PAP™ test kit. Individual bottles containing 15 mL cytopreservative solution.

FIG. 10

TRANSFER OF CERVICAL SPECIMEN FROM COLLECTION DEVICE TO THE EXAMINING MICROSCOPIC PREPARATION

Column Title Transferring device Microscopic Microscopic preparation preparation shape name

1. Pap test Spatula Smear Smear

2. LBP Cyto-Sed Medium circle Monolayer

3. LBP Cytospin; cytofunnel Small circle Monolayer

4. LBP Cytospin; Quadrangular Monolayer

5. ThinPrep Pap ThinPrep Large circle Thin-layer test PreservCyt/Processor

6. CPS Cyto-Sed Medium circle Monolayer

7. CPS Cytospin; megafunnel Quadrangular Monolayer

8. CPS Cytospin; Small circle Monolayer cytofunnel

LBP=Liquid-Based Pap; CPS=Cervical Acid Phosphatase Papanicolaou Test Solution works with all transferring devices.

FIG. 11

Table and Charts for the December 2002 Report

FIG. 12

MARK-PAP™ Test Kit's and Accessories' Individual Labels

### 7.0 BRIEF DESCRIPTION OF THE DRAWINGS

“The patent or application file contains at least one drawing executed in color. Copies of this patent or patent application publication with color drawing(s) will be provided by the Office upon request and payment of the necessary fee.”

A total of 12 drawings (figures) are included in this application at the end, but as an integral part of this Specification.

1. MARK-PAP Test Kit Applied on COMBO Control Slides.

2. MARK-PAP Test Kit Applied on Different Cervical cell Specimens: Conventional Pap smear, ThinPrep thin-layer, CPS monolayer, COMBO monolayer.

3. MARK-PAP Test Kit Applied on Cervical Specimens Collected in MARK-PAP Solution.

4. Automatic and Manual MARK-PAP Test Applied on COMBO Controls.

5. MARK-PAP Test Kit Applied on Normal and Abnormal Cervical Smears.

6. Cytology Tutorial. Images of cervical dysplasia and cervical cancer.

7. Control Papanicolaou smears: LSIL, HSIL (our material)

8. MARK-PAP Test KIT (exterior/interior view of the assembly)

9. MARK-PAP Test Solution (view of the assembly)

10. Transfer of cervical specimens from collection device to the examining microscopic preparation.

11. Table and Charts for the December 2002 Report. Evidence-based modeling of CPT monitoring. Flowchart of the slide evaluation procedure and actual data.

12. MARK-PAP Test Kit's and Accessories' Individual Labels (draft)

Figure legends are provided in the same section with the figures. More detailed description of figures is provided in the text of the Specification at appropriate sites.

### 8.0 DETAILED DESCRIPTION

The invention comprises one main embodiment (CPK) and two alternative embodiments (CPS, and CPAS). Every embodiment will be presented in two sections: Description, and Operation. Whenever possible, an example from our experience with application of the invention in practice will be added to the operation.

8.1 Main Embodiment

8.1.1 Description

The MARK-PAP™ TEST KIT (trademark) or CAP-PAP Test Kit (proprietary name), or CPK (acronym) is an assembly of reagents, controls and instructions put together to enable cytopathology laboratories to detect cervical acid phosphatase on cervical smears or monolayers of cervical cells prepared from specimens in solution

The kit is also intended to facilitate Pap test providers to perform CPT (a new, enhanced with a biomarker, Pap test) with more productivity and less liability. The prototype that is now available as CAP-PAP Test Research Kit, or CPRK, is intended for research only. It will be also used in studies designed to be supportive of an application to the FDA for market approval. (FIGS. 8 and 9)

### EXAMPLE

The following prototype has been given to a participating laboratory for an independent review:

8.1.1.1 Package (Box, Bottles, Labels)

See MARK-PAP Test at FIG. 8 (Insert with Drawings)

Kit Design

CPK Box dimensions: H: 150 mm; W: 215 mm; D: 160 mm

CPK Box content:

Three bottles 300 ml; One bottle 100 ml; One bottle 60 ml; Three dropper bottles 15 ml.

One 5-slide container for COMBO slides.

CPS Box dimensions: H: 66 mm; W: 310 mm; D: 235 mm

CPS Box content: 42 vials, 15 ml ea.

Boxes are made for BioSciCon by: Atlas Alexandria Packaging Co., Springfield, Va.

Bottles are purchased from Sigma Chem. Co.⁶⁶

Labeling

See MARK-PAP Test Kit and Accessories' Individual Labels at FIG. 12 (Insert with Drawings).

Labels presented in this picture are of actual size, they are actually used for the prototype of the kit, but their content is temporary and only for demonstration/educational purposes.

8.1,1,2 Reagents

All reagents are prepared by BioSciCon from commercially available raw material, bottled, labeled and packed for research purpose only.

Newly prepared reagents are packed in bottles and assembled in the kit box. CPK-30 is the actual kit-type number. Reagents carry on lot numbers and expiration date.

CPK 30-01

Naphthol AS-BI Phosphoric Acid Substrate Solution

15 ml dropper bottle, green cap. Final content is to be determined.

CPK 30-02

Fast Garnet GBC Solution

15 ml dropper bottle, yellow cap. Final content is to be determined.

CPK 30-03

Sodium Nitrite Solution

15 ml, dropper bottle, white cap. Final content is to be determined.

CPK 30-04

Acetate Buffer Solution.

100 ml, white bottle, white cap. Final content is to be determined.

CPK 30-05

Citrate Buffer Solution

60 ml white bottle, white cap. Final content is to be determined.

CPK 30-06

Hematoxylin Solution, Gill No. 3

300 ml bottle. Contains Hematoxylin, sodium iodate, aluminum sulfate, and a stabilizer.

CPK 30-07

Orange G-6 Solution.

300 ml opaque white bottle, orange cap. Contains Orange-G, ethanol, isopropanol, methanol, and phosphotungstic acid.

CPK 30-08

Solution EA-65

300 ml opaque white bottle, red cap. Contains Eosin Y, Fast Green FCF, Bismark Brown, phosphotungstic acid, and denatured ethanol.

Final formulation of these reagents is slightly modified in comparison with commercially available counterparts—new reagents need to have peak activity in somewhat more acid pH.

Material Needed but Not Provided

Denatured alcohol formula 3A (95% v/v ethanol and 5% v/v isopropyl alcohol); 4% buffered formaldehyde; methanol ACS grade, water-based mounting medium (e.g., glycerol gelatin); acetone ACS grade; ammonium water (freshly prepared: 0.5 ml ammonium hydroxide per 100 ml distilled water); phosphate buffered saline (PBS). Fixative is a solution containing: citrate buffered acetone, buffered formaldehyde, and/or methanol.

8.1,1,3 Controls (Combo Slides)

CPK 30-09

COMBO Control

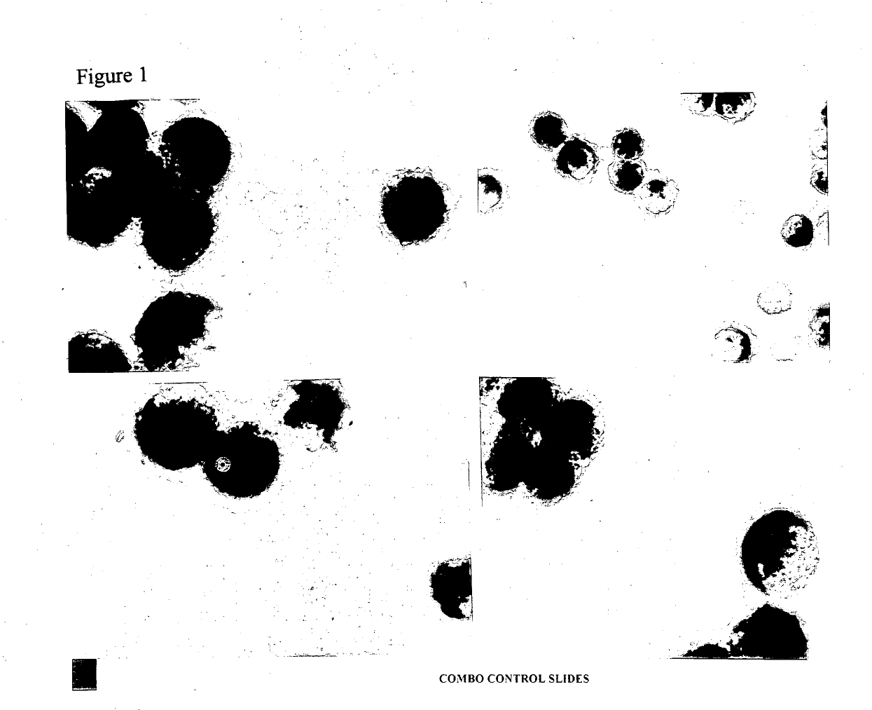

Fixed and fully CPT processed specimen of HeLa cell line and buccal cells on a microscopic slide. (FIG. 1)

CPK 30-10

COMBO Processing

Fixed, unstained HeLa cell line and buccal cells monolayers on four microscopic slides to be processed in the laboratory using CPK.

COMBO slides are prepared by BioSciCon from a mixture (1:4) of a pool of HeLa cell line in suspension (10⁶ cells/ml) and a pool of freshly prepared buccal cells (PBS suspension 10⁵ cells/ml). The HeLa cell line suspension is prepared for BioSciCon by Kemp Biotechnologies (Frederick, Md).⁵⁷

8.1.1.4 Instructions and Labeling Insert

Instructions are printed on a Labeling Insert ¹⁰⁰ added to the Kit. Detailed Instructions will be presented in a CPT Tutorial that will be prepared as a separate booklet. In summary,

The Labeling Insert describes the principle of the test, equipment and reagents, safety precautions, step-by-step technical procedure, evaluation, quality control, and references. It is designed to instruct the user procedures of how to utilize enclosed reagents and controls in order to perform CPT on Pap smears, on LBP monolayers, in automatic or manual mode, and how to perform quality control and quality assurance. This text also includes Criteria for Reporting Results of Reviewing/Screening CPT slides. (see later)

The text for the Labeling Insert is in development and is subject to adjustments due to new experiences accrued. Novel information, making distinction between this kit and similar products on the Pap test market, are presented in the Operation section of this application.

8.2 Alternative Embodiments

There are two alternative embodiments: CPS and CPAS. Although they constitute separate entities, they are included here because both are designed to work in concert with the main embodiment; with other words, CPS and CPAS are effective only when they are a part of a system including CPT.

8.2.1 Description of the CAP-PAP Test Solution (CPS)

Cervical Acid Phosphatase-Papanicolaou (CAP-PAP) Test Solution, or CPS, is any solution that can disintegrate cervical specimens into individual cells and, while keeping these cells in suspension, to protect cell integrity and cervical acid phosphatase activity for microscopic examination. CPS is intended to provide safe environments to cervical cells collected in solution, and to protect them from risks that may occur throughout the transport (doctor's office to laboratory), the storage (shelf life at room temperature), and the transfer onto microscopic slides, for staining and microscopic examination.⁷

a) CPS

CPS is a cell-enzyme-preserving solution designed to protect cervical acid phosphatase and cervical cells morphology from deterioration during a period between sampling and applying the specimen to microscopic slides for staining and further examination.

CPS is intended for collection, transport and storage of cervical specimens. There are many cell-preserving solutions available at the Pap test market. Ours is unique because it contains a unique antioxidant, ICM¹⁰ that adds for the protection of enzyme activity.

The main components of CPS are:

| Methanol | 25 | ml |
|---|---|---|
| ICM | 1 | mg |
| Inorganic salts | 0.36 | g |
| Trace metals | qs. | |
| Acetate buffer | ad 100 | ml |
| 0.1 M, pH = 5.0 | | |

CPS may contain different concentration, or different components; however, each successful combination should be able to preserve cervical acid phosphatase activity and cell morphology of cervical specimen, for at least two months storage time. The positive effect of CPS for preserving cell morphology and enzyme activity is presented on FIG. 3 (MARK-PAP Test Applied on Cervical Specimens Collected in MARK-PAP Solution) in the Insert with Drawings.

b) CPS Vial(s)

CPS is available in boxes with 42 vials of 15 ml each.

### EXAMPLE

The actual label of the CPS vial is presented on FIG. 12B.

The actual design of this accessory is presented in the Insert with Drawings on FIG. 9, MARK-PAP Test Solution.

8.2.2 CAP-PAP Test for Automatic Staining (CPAS)

a. Instrument

We have used the Varistain Gemini (ThermoShandon) Automatic Slide Stainer. On our request ThermoShandon has customized this instrument by changing its dry heated into wet heated stations. New option provided a chamber that could provide wet environment for 1 h, at 37° C.

b. Method

We had to adjust CPT manual procedure: automation required a shorter incubation time and a shorter staining time. The result was a faster, more consistent, and automatic procedure. In our parent patent application, we have described our own vision of such an automatic slide stainer with wet heated station. Cervical Acid Phosphatase-Papanicolaou Test Processor (CPP) is a name of a potential automatic slide stainer for CPT slides¹

c. Algorithm

CPT for Automatic Staining (CPAS) is a name of a group of algorithms we developed to combine our CPT with Varistain Gemini's software in an effort to use a customized (upon our specification) automatic stainer (ThermoShandon's Varistain Gemini with Wet Heated Station) for automation of CPT marker processing and staining procedures.⁹⁰

CPP_SAS-1/30 Short, is a name of a particular algorithm that provided results we used to claim an invention disclosure.⁸

For this utility patent application CPAS should be considered as “any combination of algorithms, software and Varistain Gemini with WHS (wet heated station), intended to stain gynecologic slides utilizing cervical acid phosphatase processing as one step.

### EXAMPLE

(See Operations, 9.2.2.2)

8.2.3 Other Embodiments—Accessories

The kit may be extended to include

Sampling accessories such as: extended tip spatula (n/a);

Coated microscopic slides (n/a);

Spray fixative (n/a);

Packaging and shipping material;

Cell-enzyme-protecting solution;

Instrument for cells transferring (n/a);

Software for automation;

Other invention necessary to enable the kit and CPT to be efficiently used in different situations.

NOTE: n/a denotes items in planning phase, but not in this application.

The new cell-enzyme-protecting solution and the new software for automation of CPT are described in the section Alternative Embodiments.

9.0 Operation

9.1 Main Embodiment

CPK is designed to facilitate performing CPT on microscopic slides containing cervical cells prepared as Pap smears, ThinPrep thin-layers or CPS monolayers. The liquid-based specimen collection techniques have recently been named Liquid-Based Pap technology.⁷⁸ We will use this terminology in this application.

Specimen preparation phase depends upon the source of cervical cells (Pap smear, or LBP). Specimen processing phase is unique, but is could be either manual (standard) or automatic (optional).

9.1.1 CPK for CPT

(Excerpts from Instructions)¹⁰⁰

A. Preparation of the Specimen

The Pap test provider uses one of several approved devices (long tip spatula, cervical brush, cervical broom) and techniques (one device, two devices, circumferential abrasion, local abrasion under visual control) for obtaining cervical specimen. (See FIG. 10, upper raw)

Once the specimen is on the device, it can be smeared on microscopic slide(s) or washed into a collecting solution.

The conventional Pap smear is when the specimen is smeared on a microscopic slide and immediately sprayed with any of several spray fixatives. Fixatives that contain ethyl alcohol are not suitable for CPT (inhibition of enzyme).⁹³

CPT smear obtained in doctor's office does not require spraying with fixatives. CPT specimen processing phase includes special hydration/fixation phase.⁵⁵

LBP requires washing specimen into any of commercially available cell preservative solutions and in CPS. Only PreservCyt® solution (Cytyc Cop.) developed for ThinPrep Pap Test, contains methanol and it can be used (with limitation) for CPT.

CPS contains optimal ingredients for preservation of cell morphology and acid phosphatase activity. Washing abrading devices into CPS vials is done by the same agitation technique, as described for washing other devices into other solutions.

LBP specimens must be transferred from suspension onto microscopic slides. This is the same for specimens suspended into CPS. Several techniques are available for cell transfer: (1) Artificial gravity force, (2) natural gravity force, (3) density gradient separation, and (4) filtration. All of them can be used for transferring cells suspended in CPS.

NOTE: Automatic processors, such as ThinPrep 2000 and ThinPrep 3000 cannot be used without modification.(www.thinprep.com) One of the modifications that enable the use of ThinPrep 2000 for transferring specimen from CPS onto microscopic slides, is change of the ThinPrep standard fixative with the CPT fixative (available in the CPK).⁹⁵

Different transferring devices and the appropriate techniques produce different shape of cervical samples on microscopic slides. Cytospin-2 centrifuge with Cytofunnels produces small round monolayers. Cytospin-2 with Megafunnels produce large, quadrangular shaped samples. Cyto-Sed produces medium size round shaped samples.(See FIG. 10, lower row): Sampling and transferring devices, and shapes of specimen samples on microscopic slides).

B. Specimen Processing

CPK utilization starts at the level when the specimen is prepared on microscopic slides. This procedure is described several times elsewhere^{1,54} and the Step-by-Step Procedure is described later (Section 9.1.D). In summary, the procedure begins with fixation (for monolayers) or fixation/re-hydration (for smears), it continues with the marker-processing phase (visualization of acid phosphatase activity), and cytological staining with a modified Papanicolaou staining (visualization of cell morphology), and mounting. An inherent part of this procedure is the internal and the external control performed in parallel (see below).

The entire procedure (CPT) is a single-slide, double-staining method for visualization of cervical acid phosphatase inside cervical cells visualized with standard cytological staining, thus, enabling examiners to make advanced cytological diagnosis. The original CPT (presented in the parent patent) utilized different commercially available regents; CPK provides a standard for all reagents, controls and procedures.^{1,9}

Therefore, basic difference between CPK and other Pap test-related technologies (that could also be used for CPT), is in the control of the procedure and in the materiel used.

C. Principle of the Test

Cervical acid phosphatase is not present in normal female genital epithelium. However, in pathologic conditions, in particular those which untreated may progress into cervical cancer, some biochemical changes accrue favoring occurrence of cervical acid phosphatase in squamous cervical cells. CPT identifies the presence of acid phosphatase in abnormal cervical cells; therefore, CPT identifies an increased risk for disease that may progress into cervical cancer.

The CAP-PAP test is based on the following principle: Cervical acid phosphatase (CAP) catalyzes the liberation of phosphate from a substrate α-naphthyl AS-BI phosphate. The remaining aromatic moiety of the molecule simultaneously couples with Fast Garnet GBC producing an insoluble red deposit ( enzyme product) on the sites of the enzyme activity. Counterstaining is done with a modified Papanicolaou staining procedure. CAP activity appears as a distinct brilliant-red granular deposit (marker) on the background of the Pap stained cells (blue nuclei, light blue and/or orange cytoplasm). This allows simultaneous assessment of the enzyme activity (marker) and the cellular morphology (TBS). (CPT UP, FIGS. 1-5)

However, we do not wish to be bound by this explanation of the staining procedures.

D. Step-by-Step Procedure for Marker Processing and Cytological Staining

### (EXAMPLE)

Details of CPK standard manual operation are described in the CPK Labeling Insert. This procedure has been in use since 2001, and is fully implemented in two laboratories performing CPT research in parallel with regular Pap test examination of their patients.

The technical procedure consists of Fixation, Marker Visualization, Cytological Staining with modified Papanicolaou technique, and Mounting. Run in parallel COMBO Processing slide (CPK 10-10) for quality control (QC) and quality assurance QA).

(1) Fixation

1. Prepare Fixative Solution.

d) Immerse smears in the Fixative solutions for 50 sec.

e) Rinse thoroughly in several changes of distilled water. Place slide into a staining dish for incubation.

(2) Marker Visualization

Incubate slides in the following, freshly prepared Incubation Mixture:

In a 15 ml test tube combine 10 drops Fast Garnet GBC Solution (CPK 10-02, yellow cap dropper) with 10 drops Sodium Nitrite Solution (CPK 10-03, white cap dropper). Agitate for 30 sec and allow to stand for 2.5 min at room temperature. Transfer into an Erlenmeyer flask containing 46 ml pre-warmed distilled water at 37° C., and 2.5 ml Acetate Buffer Solution (CPK 10-05, white bottle, white cap). Add 10 drops Substrate Solution (CPK 10-01, green dropper).

Incubate slides for 30-60 min at 37° C. protected from light.

Rinse slides for 5 min in running tap water, followed by rinse in distilled water. Transfer slides into the staining rack. Dip the rack with smears (10 dips) in PBS, followed by brief rinse (2 dips) in tap water pH 6.8-7.0.

Immerse slides for 2 min in Hematoxylin Solution (CPK 10-06, brown bottle) for 5 min. Rinse in running tap water for 2 min. Treat slides (6 dips) in Ammonium Water. Rinse in tap water, followed by distilled water.

(3) Cytological Staining

1. Run slides in 50%, 70%, and 95% alcohol, 6 dips each.

2. Stain with Orange-G Solution (CPL 10-08) for 3 min. Rinse in 95% alcohol, 2 changes, 6 dips each.

3. Stain with EA-65 Solution (CPK 10-08)) for 15-30 sec. Rinse in 95% alcohol, 2 changes, 6 dips each. Rinse in distilled water.

(4) Mounting

Mount in glycerol-gelatin.

(5) Results

This procedure is applicable on specimens prepared as Pap smears (directly smeared onto microscopic slides), and/or monolayers or thin-layers (transferred from cell-preservative solutions). COMBO controls are run in parallel.

Examine test and control slides with microscope objective ×20 and use ×40 for clarification. For reporting results see 9.3.1 Criteria for Reading CPT Slides, and the Color Plate.

E. Control of Specimen Processing

Internal Control and the Adequacy of Specimen Processing

Find at least one monocyte positive for acid phosphatase on CPT smears.

Find at least one endocervical cell positive for acid phosphatase on CPT monolayers.

External Control

COMBO Controls

Quality Control

COMBO slides come in a set of five; one of them is fully processed and mounted; others are to be stained with each new batch of tests. For QC purpose, the laboratory personnel will compare the results of their staining with the pre-stained slides, and will be able to decide about adequacy of their procedure, and to adjust it easily, if necessary. For the same purpose, a Gallery of Control Slides will be also included in the kit.

Compare color, size and distribution of marker pigment with HeLa cell-example on COMBO control slides (see below).

Compare quality of cytological staining and clarity of cytological images with buccal cell-example on COMBO control slides.

Quality Assurance

After processing, the COMBO slides are mounted and kept for at least three years. During this period they can always be used for

1. Comparison of CPT procedures in different laboratories;

2. Assessment of CPT processing consistency across the network of participating laboratories;

3. Evidence that CPT processing was adequate in case of liability litigations.

COMBO control slides (CCS) and the opportunity to use these standardized specimens for QC/QA is the novelty that has never before been used for Pap test.

### EXAMPLE

Please see ref. 21 “Labeling Insert.” This information is viable and is updating regularly with new incoming data.

C. Limitation and Troubleshooting

Inadequately processed specimen slides must be repeated.

Inadequately processed COMBO slides call for adjustment in the procedure.

In rare occasions when pathologist needs to investigate nuclear chromatin to be sure of cytological diagnosis, but the marker pigment is obscuring the image, it is possible to remove the pigment by distaining. This procedure might include: absolute alcohol (1 min), alcohol-xylene 10 dips, and xylene 3 min. If necessary, this treatment can be repeated.

9.1.2 Advantages

The advantages of CPK are the following:

More distinct, sharply localized, brilliant-red colored deposit, without diffusion artifacts. This color clearly contrasts the bluish Papanicolaou background counterstaining. With the conventional Sigma reagents, the color of the deposit is more brown-red, and diffusion artifacts may be seen;

Lack of non-specific staining, (e.g., substantivity), presented frequently as yellow-brownish granules throughout the cytoplasm with conventional reagents. These granules may obscure the marker, or sometimes maybe interpreted as CAP activity.

Omission of xylene in the cytological staining procedure.

Duration of the procedure, and the duration of the whole test are reduced (e.g., incubation time).

The kit further simplified the procedure, and made it even more customer friendly (e.g., use of dropper bottles, instead of using pipettes). This contributes for further reduction of the duration of the technical procedure.

The cost of the entire test is reduced.

These advantages are due to the optimization of the whole procedure for the purpose of cervical acid phosphatase visualization. Acid phosphatase isoenzyme specter differs in different cell types. Optimization was made related to the composition of incubation mixture used for enzyme visualization: The choice of the preferred substrate and its concentration, choice and the concentration of the diazonium salt mixture, diazotization, and the pH of the incubation mixture. The fixation was also optimized, in order to design the best combination for preserving both the enzyme activity and the cell morphology. For example, formaldehyde was added in low concentration to serve as a fixative, but also as an inhibitor of the erythrocyte acid phosphatase ( known to be sensitive to formaldehyde), and thus make the method easily applied on bloody specimens. These modifications, and some others, contributed to the advantages 1-4. Preparation of reagents from individual chemicals, rather then ready-made reagents, decreased the cost of test (advantage 6). The cost was also decreased by the reduction of the procedural time by the simplification of the method (advantage 4 and 5)

9.2 Alternative Embodiments

9.2.1 Operation on Specimens in Solution—CPT on LBP

Liquid-based Pap (LBP) is a new term, suggested by the American Cancer Society^{78,86} to describe the liquid-based specimen collection technology for collecting, transporting and transferring cervical specimens from sampling device to microscopic slides.

9.2.1.1.1 CPT for LBP

We have tried CPT on specimens collected in different, commercially available solutions, and we found that only PreservCyt®, the ThinPrep® Pap Test™ in cell-preservative solution, could be used for CPT. However, there is a time limitation of two weeks before enzyme begins to deteriorate. (PreservCyt contains high concentration of methanol.⁵⁵

In our manuscript CPT on ThinPrep Specimens we have described that CPT can be used for fresh ThinPrep specimens, that acid phosphatase on thin-layers is distributed similarly as on Pap smears, that internal control on thin-layers are endocervical cells (monocyte on Pap smears), and that sensitivity of detecting abnormal slides with CPT was equal if not better than the original ThinPrep screening efficacy. For details see Appendix).⁹⁴

We have used ThinPrep slides as standard for assessing the efficacy of CPS for providing specimens for CPT (BSC-0110, and BSC-0111).⁹⁵

9.2.1.2 CPS for CPT

### EXAMPLE

CAP-PAP Test Solution (CPS) is described above as a tool for collecting specimens in a cell-enzyme-protective media, to assure specimen protection from collection, throughout transport and storage, to the final stage—transfer of specimen onto microscopic slides

9.2.1.2.1 Procedure

Although different procedures are possible, we are describing one that is in effect in our studies.

| Step | Procedure | Instrument | Activity | Comment |
|---|---|---|---|---|
| 1 | Sampling | Extended tip | Circumferential | T zone cells |
| | | plastic spatula | abrasion | |
| 2 | Smearing | Microscopic | Sample smearing | |
| | | slide | | |
| 3 | Liquid- | CPS vial | Washing spatula | |
| | collection | | from cells | |
| | | | inside | |
| | | | the vial | |
| 4 | Transport | Packaging | Vials are | |
| | | material | packed as | |
| | | | hazardous | |
| | | | material | |
| 5 | Storage | Refrigerator | Suspension | |
| | | (4-10° C.) | protects for | |
| | | | 4-6 weeks | |
| 6 | Transfer | Cytospin 2 | Put suspension | Use only coated |
| | | | in megafunnels, | slides to keep |
| | | | centrifuge at | cells on slides. |
| | | | 400 rpm for | |
| | | | 5 min | |
| | | Cyto-Sed | Place suspension | |
| | | | into wells and | |
| | | | let sedimentation | |
| | | | to occur for 1 h | |
| 7 | Fixation | Fixative | Fix slides for | CPK contains |
| | | | 50 sec in CPK | reagents. |
| | | | fixative | Fixative must |
| | | | | be prepared ex |
| | | | | tempore |
| 8 | Referral | Marker | Fixed specimen | |
| | | processing | can be stored for | |
| | | unit | at least two weeks | |

CPS is packaged in vials (42 vials, 15 ml solution ea). CPS vials are distributed to Pap test providers. (FIG. 9)

9.2.2 Automation of CPS

Automation of CPS procedure include (1) Using instruments for transfer of cells from suspension onto microscopic slides, and (2) automatic slide staining.

9.2.2.1 Transfer of Specimens from Solution onto Microscopic Slides

Cervical cells can be transferred from suspension onto microscopic slides via gravity force (natural or artificial) , filtration, or combination. Depending upon the transferring device, final distribution of cells on slides (monolayer, thin-layer) can take round or quadrangular form. Each of these monolayers has a pre-measured size, and an approximate number of cells. Our test has been tested on all of them, and all are applicable. (See FIG. 10)

Upon our experience, the best device should be able to produce a monolayer with at least 5-10,000 individual cells that could be screened at microscope magnification of ×20 for about 3 min per slide.

a. Artificial Gravity Force.

Any device that can produce gravity force equivalent to 400 rpm for 5 min, and can be used for transfer of cells onto microscopic slides.

Any device that combines a funnel and a microscopic slide in a centrifuge attachment, and any centrifuge that has a head with connectors for these attachments, can be used for transfer of cervical cells suspended in CPS onto microscopic slides.

### EXAMPLE

We have used, across the studies, the Cytospin-2 centrifuge (ThermoShandon, Pittsburgh, Pa.), cytofunnels (alternative megafunnels), and coated microscopic slides. (See FIG. 10)

b. Natural Gravity Force.

Any device that can use the natural gravity force to sediment cells from LBP onto microscopic slides as to make a monolayer of cervical cells covering a definitive area (round or quadrangular), may be used for transfer of cervical cells suspended in CPS onto microscopic slides.

### EXAMPLE

We have used, in one study, the Cyto-Sed (Surgipath, Richmond, Ill.). This device utilizes natural gravity force for one hour to sediment cells from the suspension onto microscopic slides while the solution is either evaporated from the well or diffused into the filter paper surrounding the opening.

c. Filtration

Filtration is the working principle for the ThinPrep Processor. The suspension is forced through a filter: debris and small cells are removed while large cervical cells are retained on the filter. In a second act, those retained cells are transferred onto microscopic slide and fixed. A result is a clean thin-layer of cervical cells, easy for examination and very much appealing to pathologists. However, the procedure can miss many cells that might have diagnostic value, in particular small mammal cells. The cost if processing device and filters makes the use of this system very costly and, unless the cost is reduced, we would not recommend using this technique for CPT. (www.thinprep.com)

9.2.2.2. Automatic Slide Staining (Specimen Processing)

In this section we will describe CPT processing when kit reagents are used to stain slides with specimens using a customized automatic slide stainer (Varistain Gemini with wet Heated Station, ThermoShandon, Pittsburgh, Pa.). (www.thermoshandon.com)

FIG. 4 BSC-0003 Auto vs. manual (table, picture, results)

CPP_SAS-1/30: Software for Automatic Staining of CAP-PAP Test, was disclosed to USPTO in October, 2001 (DD#504,234).⁸ This procedure is on the table First Algorithm.

a) First Algorithm (CPP_SAS-1/30)

CPP_SAS-1/30 Short Algorithm for Automation of CAP-PAP Test

| No. | Period | Station | Reagent | % | Uses | Time | Limit | Agitate |
|---|---|---|---|---|---|---|---|---|
| 1 | A | A | Dry storage | | X | Load | No max | None |
| | | | | | | 00.00 | | |
| 2 | F/R | 1 | Sol F | )* | 10 | 00:50 | Critical | None |
| 3 | F/R | 2 | DW 1 | | 10 | 03:00 | Standard | Frequent |
| 4 | F/R | 3 | DW 2 | | 10 | 03:00 | Standard | Frequent |
| 5 | MP | WHS | Sol I | )* | 10 | 30:00 | No max | None |
| 6 | MP | RW | Running | /* | | 05:00 | Standard | Continuous |
| | | | water | | | | | |
| 7 | MP | 21 | DW 3 | | 10 | 00:20 | Standard | None |
| 8 | MP | 22 | Sol B | )* | 10 | 00:40 | Standard | Continuous |
| 9 | MP | 25 | Tap | /* | 10 | 00:30 | Standard | None |
| | | | water 1 | | | | | |
| 10 | MP | 24 | Sol H | )* | 10 | 03:00 | Critical | None |
| 11 | MP | RW | Running | /* | | 03:00 | Standard | None |
| | | | water | | | | | |
| 12 | MP | 23 | Sol W | )* | 10 | 00:15 | Critical | None |
| 13 | MP | 18 | Tap | /* | 10 | 00:30 | Standard | None |
| | | | water 2 | | | | | |
| 14 | MP | 26 | DW4 | | 10 | 00:30 | Standard | Continuous |
| 15 | MP | 4 | Alcohol | 50 | 10 | 00:40 | Standard | Continuous |
| 16 | S | 5 | Alcohol | 70 | 10 | 00:40 | Standard | Continuous |
| 17 | S | 6 | Alcohol | 80 | 10 | 00:40 | Standard | Continuous |
| 18 | S | 8 | Alcohol 1 | 95 | 10 | 00:40 | Standard | Continuous |
| 19 | S | 9 | Sol P1 | | 10 | 03:00 | Critical | None |
| 20 | S | 10 | Alcohol 2 | 95 | 10 | 00:40 | Standard | Continuous |
| 21 | S | 11 | Alcohol 3 | 95 | 10 | 00:40 | Standard | Continuous |
| 22 | S | 12 | Sol P2 | | 10 | 00:15 | Critical | None |
| 23 | S | 13 | Alcohol 4 | 95 | 10 | 02:00 | Standard | Continuous |
| 24 | S | 15 | Alcohol 5 | 95 | 10 | 02:00 | Standard | Continuous |
| 25 | S | 16 | DW 5 | /* | 10 | 02:00 | Standard | Continuous |
| 26 | End | D | DW 6 | /* | 10 | Unload | No max | None |
| | | | | | | 30:00 | | |

)* = Staining solutions are previously described
/* = Wash/Rinse

b) New Algorithms

With expansion of our research interest we change composition of reagents, change procedures and goals. Each change has been tested in a manual and an automatic mode. Slight changes of automatic procedure had to be made for each of them. A family of CPAS programs has been developed, and new will be added. This was made possible by introducing into the ThermoShandon instrument and our test the following novelties:

1. Wet Heated Station (WHS).

The original Gemini automatic stainer has Dry Heated Station. We have requested, and ThermoShandon produced a custom made instrument with WHS. This special addition to the stainer, has enabled the automatic processing of all enzymatic reactions requiring 30° C.

2. Algorithm SAS-1/30.

This algorithm was inserted into the Shandon's software. It is unique because it combines CPT and Gemini WHS, two also unique entities: a new test for continuous automatic marker processing and cytology straining, and a prototype of a new machine.

Automatic versus manual slide staining (from the BSC-0003 study report)

The BSC-0003 was conducted as a part of the SBIR Phase-2 research project, “CAP-PAP Test for Cervical Cancer Screening.” In this study, we have studied acid phosphatase score between HeLa cell line monolayers processed manually and automatically, and between COMBO monolayers processed the same way.

When the new software was used for automation, we were not able to see difference in the quality of images from specimens stained with the standard manual or the new automatic mode. (See FIG. 4, Automatic and Manual MARK-PAP Test Applied on COMBO slides).¹⁰⁰

In both experiments we used random pair design, 30 slides at each group, and a semi-quantitative estimate of enzyme activity—the score technique. Results were compared using 95% and 99% confidence intervals for difference between means in paired groups. In both experiments zero was inside the interval, meaning that true difference probably does not exist. We concluded that the suggested algorithm, if used for slide automatic slide staining on Gemini WHS, would produce the same results as manual staining; therefore, the advantages of automation (reduction of human work, speed, and consistency) became more obvious.¹⁰⁰

9.3 CPT Criteria for Reporting Results of Screening

CPT slides differ from Papanicolaou stained slides only by having a new cytological marker for identification of abnormal squamous cells. Therefore, we had only to extend cytological criteria developed in the current classification (2001 Bethesda System). The additional criteria are summarized bellow:

9.3.1 Criteria for Reading CPT Slides

(Color Plate)

a) Basics:

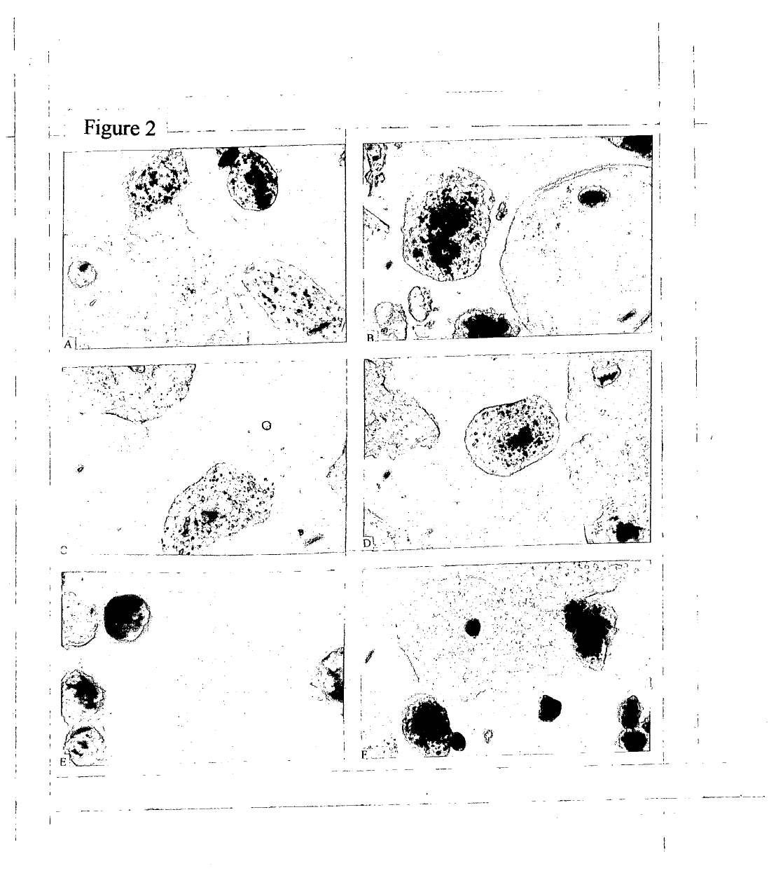

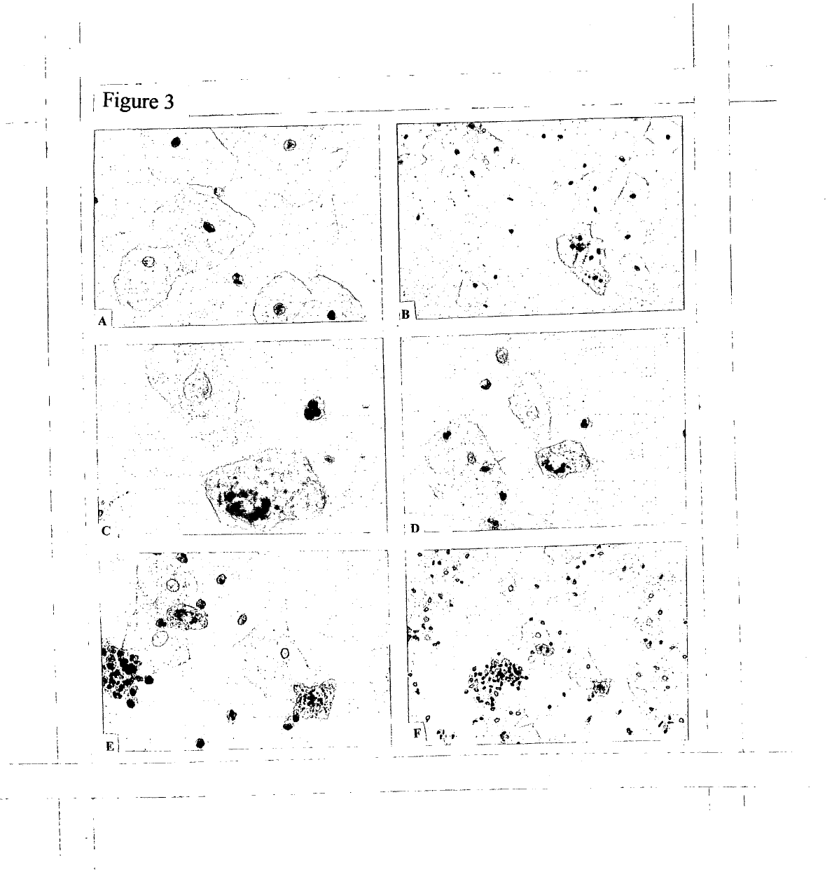

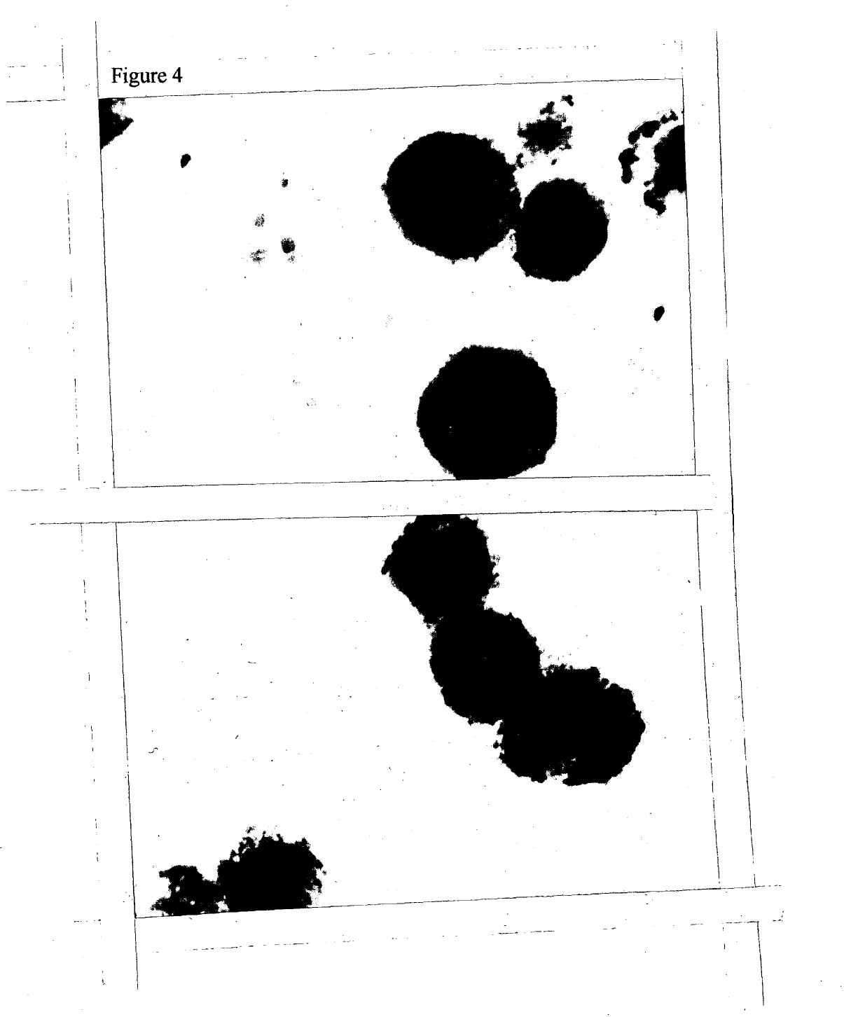

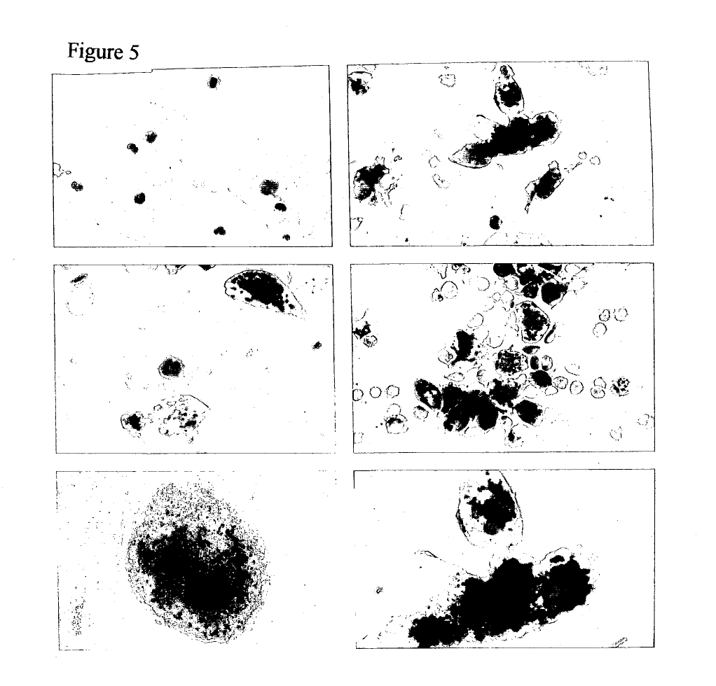

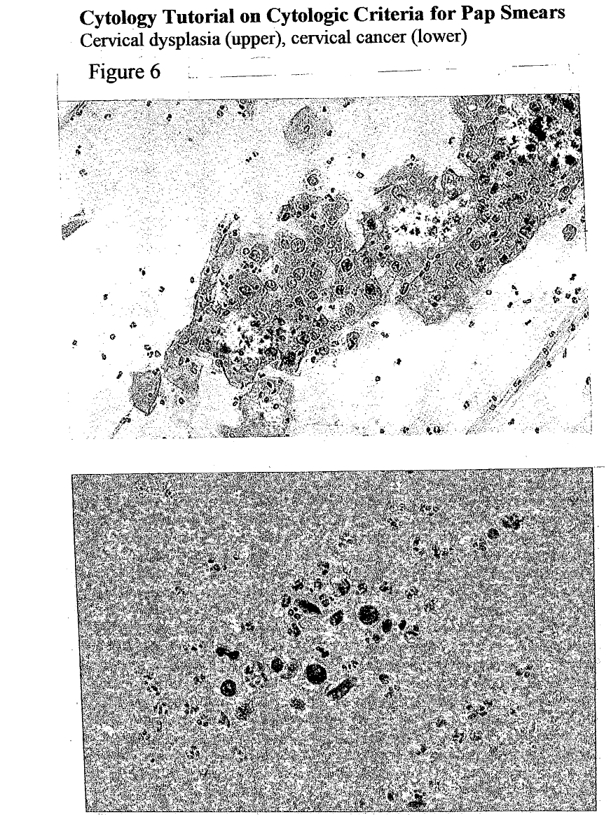

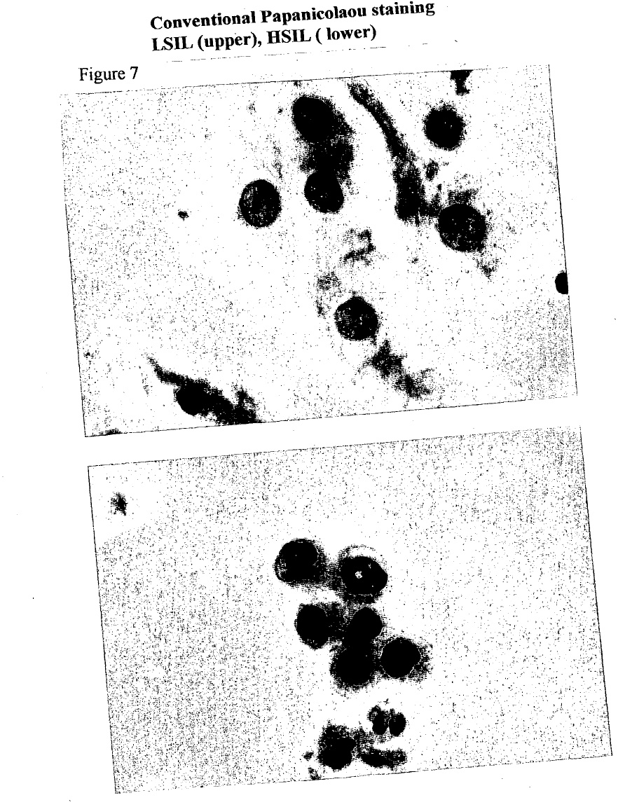

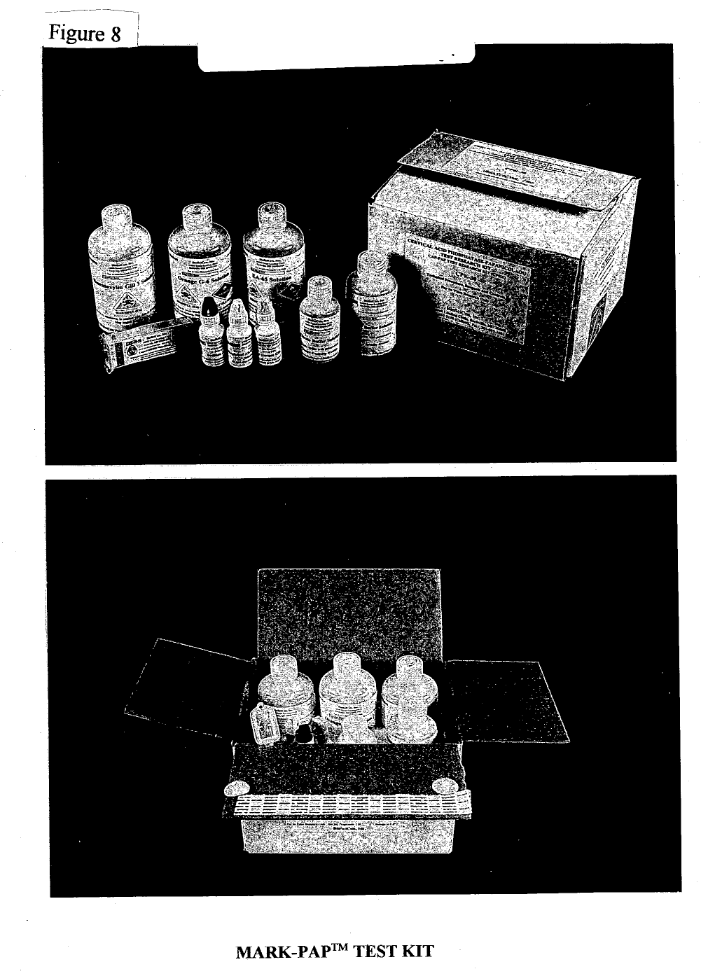

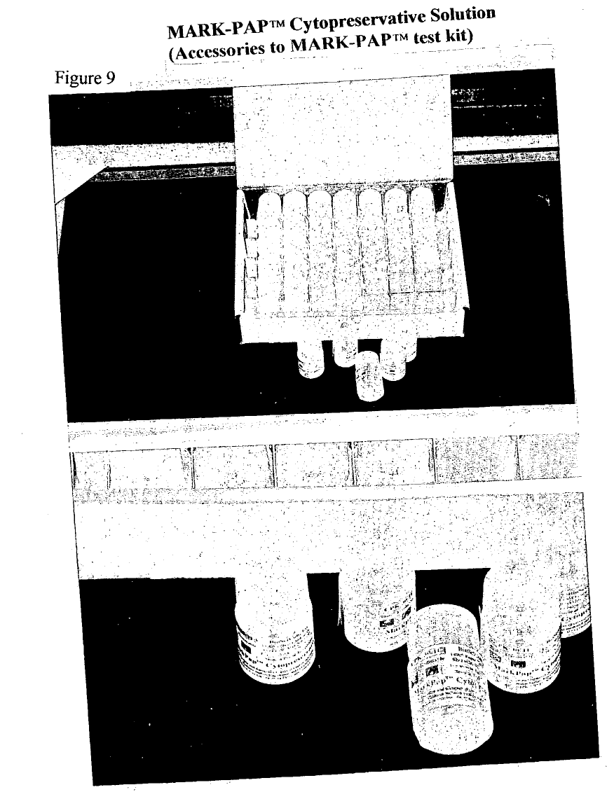

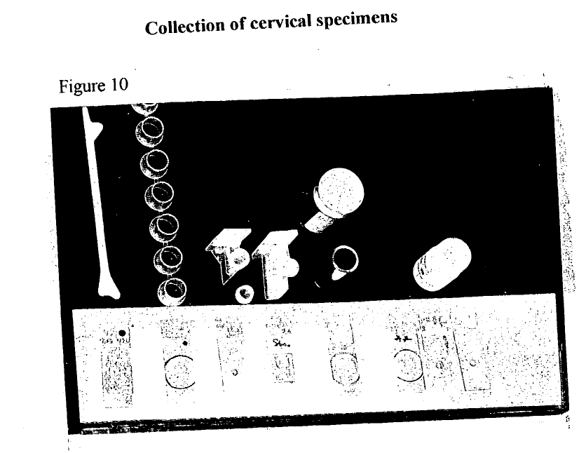

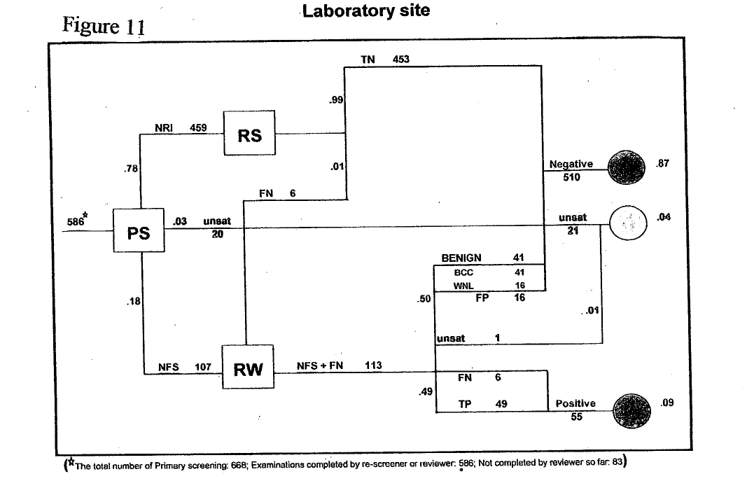

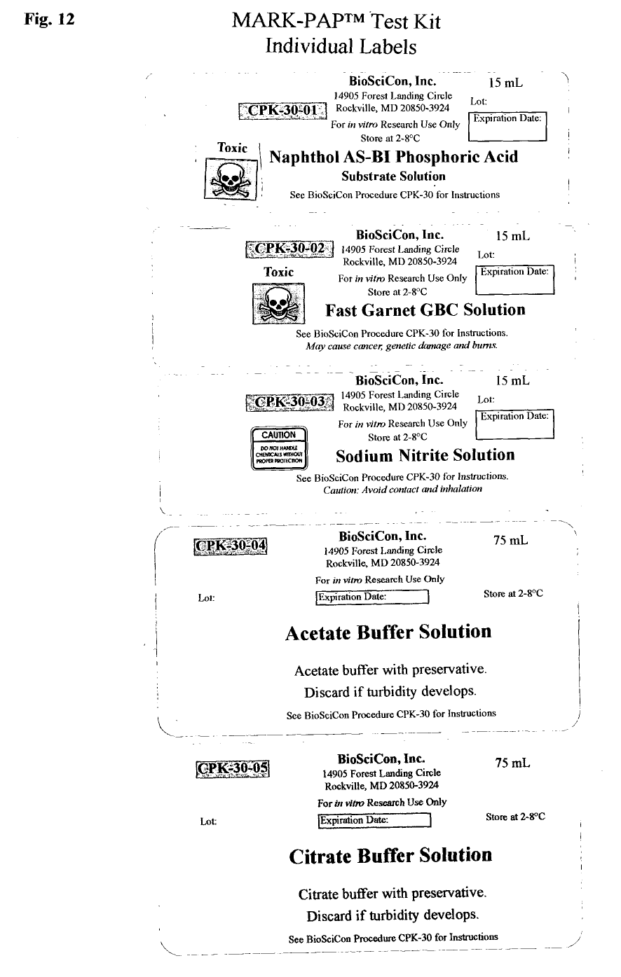

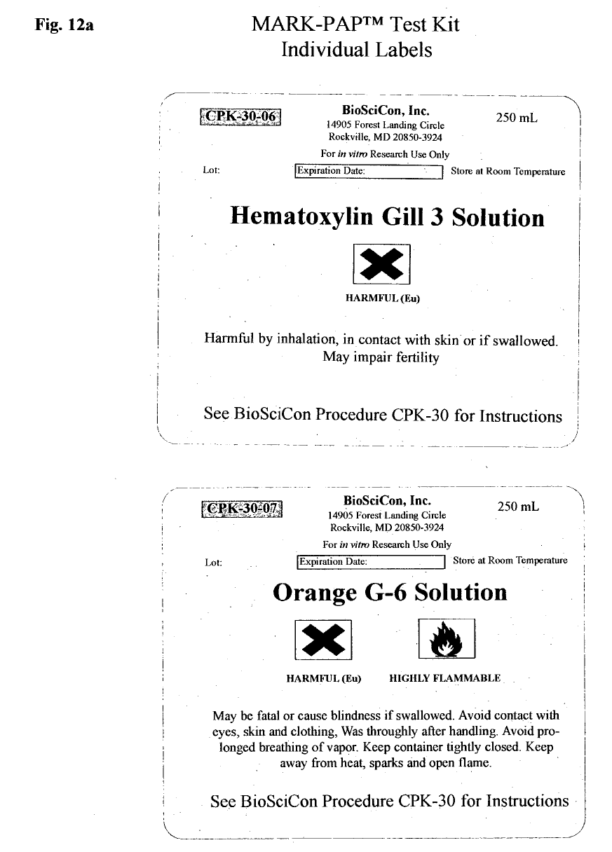

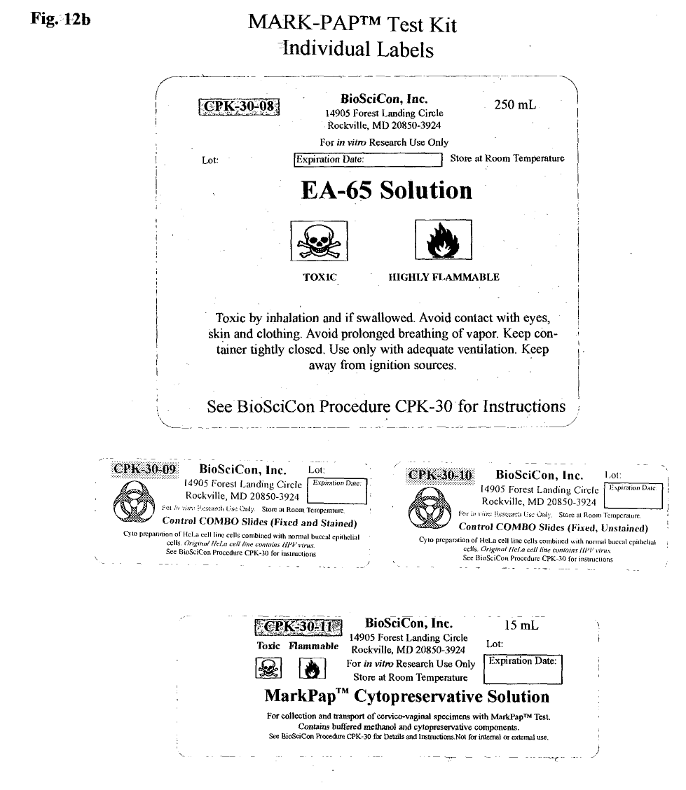

CAP is present in abnormal squamous cells, endocervical/endometrial cells, some metaplastic cells, monocytes and rarely in neutrophils. (FIGS. 2,3 and 5)

CAP is always absent in normal squamous cells. (FIGS. 2, 3 and 5)

b) Adequacy of Staining:

CAP positive monocytes on Pap smears.(FIG. 2)

CAP positive endocervical cells on monolayers. (FIG. 3)

c) Result:

CAP positive squamous cells (one or more) found at primary screening or rescreen) qualifies the specimen for review by pathologist. (FIGS. 2, 3 and 5)

Only pathologist gives cytology diagnosis. (FIGS. 6 and 7)

Verified staining adequate slides with squamous cells lacking acid phosphatase activity will be reported as CPT negative/normal. (FIG. 3). (See FIGS. 6 and 7, Cytology Tutorial link for comparison with standards).

9.3.2 Recommended Procedure for Screening CPT Slides

For screening purposes, the examiners are interested in cervical cells that can help them reach decision on whether the examined specimen contains signs of precancerosis/cancer, and what next to be recommended to the specimen donor.

We recommend that each slide (positive or negative) be seen by at least two screeners. The following flowchart has been developed for users of CPT.

See FIG. 11 Table and Charts of the December 2002 Report.⁹⁹ This diagram presents our evidence-based model system where we follow decisions made at the level of primary screening (PS; 100% all slides), rescreen (RS; 100% normal/negative slides) and review (RW; 100% abnormal/positive slides). Note the low false-negative rate 0.02, and the doubled detection of abnormal slides (0.10 vs. 0.05).

This system is possible because of the short screening time required for examining CPT slides. Indeed, CPT is solving two problems: (provide means for 2-level primary screening for both normal and abnormal slides, and provides reason to Federal authorities to change the 10% requirement for rescreen on normal slides, and to affirm their policy of 2-level screening. This policy change, together with out kit and accessory solution should reduce false negatives to less than the College of American Pathologists believed is possible.⁷⁶ CPT and CPK bring this achievement because they use a selective biomarker of cervical cell abnormality IN ADDITION to the standard cytology for diagnosis of clinical condition on slides. CPT, indeed, has one more parameter for assessment of cell abnormality. This parameter, acid phosphatase is present only in abnormal cervical squamous cells. This way. The conventional Papanicolaou staining is improved—it has been neither achieved not even challenged with all technologies that occurred after the 1996 NIH Consensus Conference on Cervical Cancer, and their call for improvement of Pap test.⁸⁹

a) Primary Screener:

1. Procedure adequacy:

Requires one positive monocyte on Pap smears or one positive endocervical cell (EC) on LBP smears. This is utilized as the internal quality control for adequacy of staining.

2. Preliminary decision on clinical condition:

Requires one positive squamous cell for sorting this slide into the abnormal cell category that should be reviewed by a pathologist.

3. If no positive squamous cells are found, the slide is categorized as normal/negative and it is referred to another cytotechnologist for rescreen.

4. Average time for screening 100 CPT slides is 5 h, or 3 min per slide.

b) Rescreen

Searching for red marker over the slides, verifies the primary screener decision on ALL negative slides.

1. If a single squamous cell is found CAP positive, the slide is re-categorized into false negative (FN) class and referred to the pathologist for review.

2. If the marker labels only internal controls (monocytes on Pap smears, EC on LBP), no squamous cell is positive, and other labeled cells (metaplastic, EC) do not exceed the number considered as normal, verifies the primary screening decision and classifies the slide to be true negative/normal.

3. This decision is considered final for the laboratory result.

4. Rescreen requires an average screening time of 1 min per slide, or less than two hours for 100 negative slides.

c) Reviewer

Verifies the results of primary screening of ALL positive/abnormal slides, and those referred by the rescreener as false negative.

Using the 2001 Bethesda System classification⁷⁸ categorizes the clinical condition on the slide into categories: NIL (negative for intraepithelial lesions) includes former category WNL and BCC (normal with secondary benign changes), or SIL (squamous intraepithelial lesion) including categories: ASC-US, ASC-H, LSIL, HSIL, and cancer; or GIL (glandular intraepithelial lesion) including categories AGC (atypical glandular cells), and AIS (adenocarcinoma in situ).

If the slide is classified into NIL the primary screening decision is found to be false positive.

If the slide is classified into ASC-US or upper categories, the primary screening decision is verified.

This decision is considered final for the laboratory result.

d) Reporting Results

1. Verified negative/normal slides are reported as CPT negative.

2. Verified positive/abnormal slides are reported as CPT positive test.

3. If decision is not made, the report should be CPT equivocal test.

9.3.3 Recommended Clinical Actions

1. All CPT negative subjects are recommended for the next regular Pap test check-up.

2. All CPT positive patients are recommended for colposcopy as the next screening step, but the first diagnostic procedure for clarification of the clinical condition.

NOTE: More clinical experience is necessary to refine these criteria and recommendations. At present they are used for research purposes only.

10.0 Future Development

CPK is now for research purposes only. A prototype of this kit CPRK (CAP-PAP Test Research Kit) has been assembled and it is in a process of validation.

Replicas of this prototype are in use in clinical laboratory trials. CPRK and results of technical and clinical validation will be offered to FDA for approval under a new name CPDK (CAP-PAP Test Diagnostic Kit) or the trademark Mark-Pap Kit (MPT). Only slight changes, if any, might be expected, but they should not alter any of the invention disclosed in this patent application.

We claim:

1. The method and the kit therefore as:
An assembly of any reagents, controls and procedures put together to serve as a tool for demonstration of acid phosphatase inside cervical cells smeared on microscopic slides or prepared as thin- or mono-layers, and
Any set of criteria for interpretation of papanicolaou staining-based cytology with cervical acid phosphatase as a biomarker of cervical cells abnormality, and determination of clinical condition based on these criteria on cervical cell specimens obtained from healthy or sick women;
Any individual reagent included in said kit;
Any control slide included in said kit, in particular COMBO controls slides, which are mono-layers of hela and buccal cells transferred on microscopic slides (with any of cell transferring techniques) in order to serve as tools for quality control and/or quality assurance of successful demonstration of cervical acid phosphatase on cervical specimens;
Any criterion described in said set of criteria for combining papanicolaou staining-based cytology with cervical acid phosphatase biomarker for cervical cancer screening, prevention, detection or diagnosis of cervical dysplasia or cervical cancer;
Any procedure (described in said kit “Labeling Insert”) that combine said reagents and said controls into a unique mechanism for demonstration of cervical acid phosphatase as a new biomarker of cervical cancer or precancerous clinical conditions on cervical specimens.

2. The kit accessories, in particular the MARK-PAP (CAP-PAP) Test Solution for collecting cervical specimens and their transport to laboratory for examination,
Any composition of said solution, in particular when the active ingredients such as methanol is in concentration between 20% to 55%, acid buffer in pH range between 3.5 and 6.0, and ICM (fructosehexanoate, beta-cyclodextin), an antioxidant in concentrations from 10 to 100 micrograms per ml;
The use of said solution in any other indication (e.g., blood banking, cell culture, stem cell production) requiring cell-preservation for specimen collecting, transport or storage;
The use of said antioxidant (ICM) in any other indication (e.g., blood banking, cell culture, stem cell production) requiring cell-preservation for specimen collecting, transport or storage.

3. Any use of said kit (in particular the use of said biomarker and said controls) and said accessory solution for demonstration/visualization of cervical acid phosphatase for any purpose, but particularly for cervical cancer screening, prevention, detection or diagnosis of cervical dysplasia, cervical cancer or any other disorder (inflammatory [e.g., HPV infection], degenerative, idiopathic) that, if not treated and/or spontaneously cured, may progress into cervical cancer; and
Any use of said kit, said method, said reagents an/or said procedures on automatic slide stainers, image analysis-based computer assisted slide examining and interpreting instruments.
